(19) United States
(12) Patent Application Publication
(10) Pub. No.: US 2024/0423699 A1
(43) Pub. Date: Dec. 26, 2024

(54) MEDICAL SYSTEM AND METHOD FOR DETECTING CONTACT STATE

(71) Applicant: OLYMPUS CORPORATION, Tokyo (JP)

(72) Inventors: Kantaro NISHIKAWA, Tokyo (JP); Hisatsugu TAJIMA, Tokyo (JP)

(73) Assignee: OLYMPUS CORPORATION, Tokyo (JP)

(21) Appl. No.: 18/533,826

(22) Filed: Dec. 8, 2023

Related U.S. Application Data
(60) Provisional application No. 63/522,750, filed on Jun. 23, 2023.

(51) Int. Cl. A61B 18/14; G06T 7/246

(52) U.S. Cl. CPC A61B 18/1445; G06T 7/246; A61B 2018/00601; A61B 2018/00779; A61B 2018/00904; G06T 2207/10068; G06T 2207/20081; G06T 2207/30004

(57) ABSTRACT

A medical system includes a memory which stores a trained model, and a processor. The trained model is a model trained by training data including an endoscopic image and a contact state between a treatment tool and a tissue in the endoscopic image. The endoscopic image is captured by an endoscope which captures an image including the treatment tool and a tissue to be treated. The processor acquires the endoscopic image including the treatment tool and the tissue. The processor uses the trained model to detect, from the endoscopic image, the contact state between the treatment tool and the tissue as first information.

### CROSS REFERENCE TO RELATED APPLICATIONS

This application is based upon and claims the benefit of priority to U.S. Provisional Patent Application No. 63/522,750 filed on Jun. 23, 2023, the entire contents of which are incorporated herein by reference.

### BACKGROUND OF THE INVENTION

Japanese Unexamined Patent Application Publication No. 2003-61979 discloses a medical equipment which detects contact between a treatment tool and an object by a tactile sensor in an endoscopic procedure. This medical equipment is used in combination with an endoscope, including an insertion section to be inserted in a body cavity and a tactile sensor provided in the insertion section to be able to detect contact of an object with the insertion section outside an observational field of view of the endoscope.

### SUMMARY OF THE INVENTION

In accordance with one of some aspect, there is provided a medical system comprising:
a memory that stores a trained model trained by training data, the training data including an endoscopic image captured by an endoscope that captures an image including a treatment tool and a tissue to be treated, and a contact state between the treatment tool and the tissue in the endoscopic image; and
a processor.
wherein the processor is configured to:
acquire the endoscopic image including the treatment tool and the tissue; and
detect, from the endoscopic image, the contact state between the treatment tool and the tissue as first information by using the trained model.

In accordance with one of some aspect, there is provided a method for detecting a contact state, comprising:
capturing an endoscopic image including a treatment tool and a tissue to be treated by an endoscope;
detecting, from the endoscopic image, the contact state between the treatment tool and the tissue as first information using a trained model trained by training data, the training data including the endoscopic image and the contact state between the treatment tool and the tissue in the endoscopic image.

In accordance with one of some aspect, there is provided a medical system comprising:
a memory that stores a trained model trained by training data, the training data including an endoscopic image captured by an endoscope that captures an image including a treatment tool with an openable/closable jaw and a tissue to be treated, and an open/closed state of the jaw in the endoscopic image; and
a processor,
wherein the treatment tool is an energy device that treats the tissue by outputting energy from the jaw, and
the processor is configured to:
acquire the endoscopic image including the treatment tool and the tissue;
perform first detection that detects opening/closing of the jaw from the endoscopic image using the trained model;
perform second detection that detects presence/absence of a tissue between the jaw based on electrical information on the energy output; and
detect, based on results of the first detection and the second detection, a grasped state of the tissue by the jaw as the contact state.

In accordance with one of some aspect, there is provided a method for detecting a contact state that detects a contact state between a treatment tool and a tissue to be treated, the treatment tool having an openable/closable jaw and treating the tissue by outputting energy from the jaw, the method comprising:
capturing an endoscopic image including the treatment tool and the tissue by an endoscope;
performing first detection that detects opening/closing of the jaw from the endoscopic image using a trained model trained by training data, the training data including the endoscopic image and an open/closed state of the jaw in the endoscopic image;
performing second detection that detects, based on electrical information on the energy output, presence/absence of a tissue between the jaw; and
detecting, based on results of the first detection and the second detection, a grasped state of the tissue by the jaw as the contact state.

### BRIEF DESCRIPTION OF THE DRAWINGS

FIG. 1 is an example of support provided by a medical system to a practitioner.

FIG. 2 is an example configuration of a medical system.

FIG. 3 is an example flow of processing performed by a medical system.

FIG. 4 is a first example configuration of a treatment tool detection section and a contact detection section.

FIG. 5 is a second example configuration of a contact detection section.

FIG. 6 is a diagram illustrating movement vectors of a treatment tool and a tissue.

FIG. 7 is a diagram illustrating “distance difference between treatment tool and tissue in movement direction” obtained by a preprocessing section.

FIG. 8 is a diagram illustrating “distance difference between treatment tool and tissue in movement direction” obtained by a preprocessing section.

FIG. 9 is a diagram illustrating “direction difference between treatment tool and tissue in movement direction” obtained by a preprocessing section.

FIG. 10 is a diagram illustrating “statistics of movement amount distribution of tissue obtained by change of basis based on movement direction of treatment tool” obtained by a preprocessing section.

FIG. 11 is a diagram illustrating “statistics of movement amount distribution of tissue obtained by change of basis based on movement direction of treatment tool” obtained by a preprocessing section.

FIG. 12 is a diagram illustrating “color information” acquired by a preprocessing section.

FIG. 13 is a first example of a setting of a region of interest.

FIG. 14 is a second example of a setting of a region of interest.

FIG. 15 is a third example of a setting of a region of interest.

FIG. 16 is the third example of a setting of a region of interest.

FIG. 17 is a fourth example of a setting of a region of interest.

FIG. 18 is the fourth example of a setting of a region of interest.

FIG. 19 is a diagram illustrating detection of cutting apart of a tissue.

FIG. 20 is a diagram illustrating detection of cutting apart of a tissue.

FIG. 21 is a first example configuration of a contact detection section in a second embodiment.

FIG. 22 is a diagram illustrating a second example configuration of a jaw open/close detection section.

FIG. 23 is a second example configuration of a tissue between jaws detection section.

FIG. 24 is a third example configuration of a tissue between jaws detection section.

FIG. 25 is a fourth example configuration of a tissue between jaws detection section.

FIG. 26 is a second example configuration of the contact detection section in the second embodiment.

FIG. 27 is an example of contact detection using a detection result stored in a memory.

FIG. 28 is a third example configuration of the contact detection section in the second embodiment.

FIG. 29 is a first example configuration of a contact detection section in a third embodiment.

FIG. 30 is a first example flow of processing performed by the contact detection section in the third embodiment.

FIG. 31 is a second example flow of processing performed by the contact detection section in the third embodiment.

FIG. 32 is a second example configuration of the contact detection section in the third embodiment.

FIG. 33 is an example configuration of a contact detection section in a fourth embodiment.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Technique

In a procedure using an endoscope and a treatment tool, it is preferable that a medical system can detect a contact state between the treatment tool and a tissue and present information to a practitioner based on a detection result thereof. Although there are various situations that require such contact detection, described herein is an example in which support information is presented to a practitioner in treatment using an energy device.

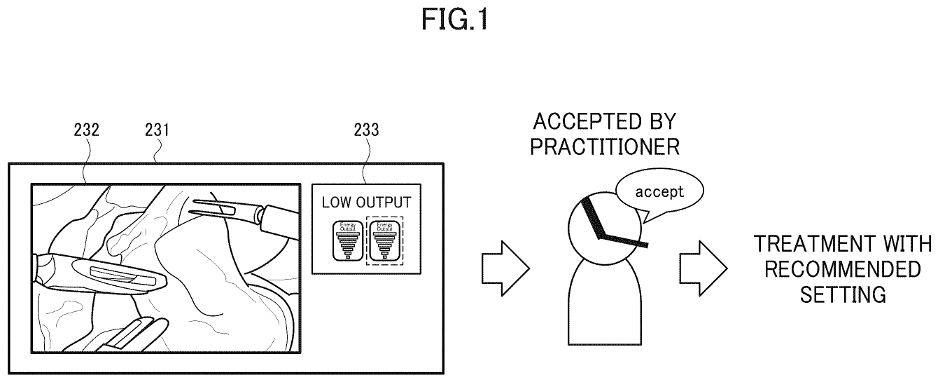

FIG. 1 is an example of support provided by a medical system to a practitioner. The medical system displays an endoscopic image 232 on a monitor 231. The medical system acquires information about a treatment target tissue from the endoscopic image 232, determines suitable energy output according to the information, and displays an output setting 233 thereof on the monitor 231 to present it to the practitioner. The practitioner accepts or rejects the output setting 233 recommended by the medical system. When the output setting 233 is accepted, the medical system outputs energy with the output setting 233 from an energy device.

Such automatic adjustment of the energy output can improve safety of treatment. However, constant presentation of the recommended output setting 233 to the practitioner is annoying for the practitioner. Hence, it is preferable to present the information when the practitioner intends to perform the treatment. For example, it is preferable to present the information when the treatment tool comes in contact with the tissue or a forceps-type surgical treatment tool grasps the tissue. To allow information presentation at appropriate timing, it is necessary to accurately recognize a contact state between the treatment tool and the tissue.

Accordingly, the medical system of the present embodiment detects the treatment tool from the endoscopic image and recognizes, based on the detection result, the contact state between the treatment tool and the tissue. Alternatively, the medical system combines a plurality of machine learning models, or combines a machine learning model and a non-machine learning technique, thereby improving accuracy of contact detection. This allows presentation of support information such as information about the contacted tissue to the practitioner only when the treatment tool comes in contact with the tissue, thereby preventing information presentation at unnecessary timing for the practitioner and reducing annoyance.

While the tactile sensor is installed on the treatment tool in the Japanese Unexamined Patent Application Publication No. 2003-61979 described above, the medical system of the present embodiment can detect the contact state between the treatment tool and the tissue by recognition processing using an image. This makes it possible to recognize the contact state without increasing the size of the treatment tool while reducing a device cost. In addition, the disclosure of the Japanese Unexamined Patent Application Publication No. 2003-61979 described above is limited to a device equipped with a tactile sensor, the technique of the present embodiment does not limit a device. In other words, the technique of the present embodiment is applicable to a variety of existing treatment tools.

An example of the contact state detected in the present embodiment will be provided. The contact state is that the treatment tool is in contact with the tissue, or the treatment tool is not contact with the tissue. In addition, the contact state may be that the treatment tool is grasping the tissue, or the treatment tool is not grasping the tissue. When the treatment tool is grasping the tissue, it is considered that the treatment tool is in contact with the tissue. The contact state may also be transition of a state from contact to non-contact, from non-contact to contact, from grasp to non-grasp, or from non-grasp to grasp. For example, cutting apart of the tissue described below corresponds to the transition of the state from contact to non-contact or from grasp to non-grasp. Further, the contact state may be a state of maintained contact, maintained non-contact, maintained grasp, or maintained non-grasp. Further, the contact state may be a combination of one or more of the above states. Further, the treatment tool includes a shaft and an end effector provided on a distal end of the shaft to treat the tissue. In this case, the contact state to be detected is the contact state between the end effector and the tissue. However, when it is more preferable that the shaft is not contact with the tissue, for example, the contact state between the shaft and the tissue may be detected.

A time period or timing at which the contact state is detected may be arbitrary. For example, when the treatment tool is an energy device, the contact state may be detected when no energy is output, or during energy output, or during both.

### 2. First Embodiment

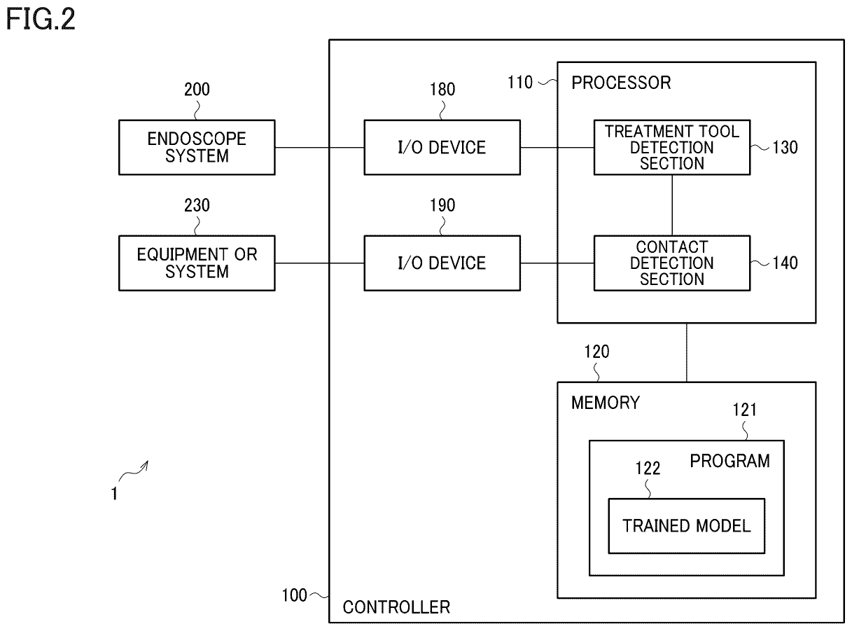

FIG. 2 is an example configuration of a medical system. A medical system 1 includes an endoscope system 200, a controller 100, and an equipment or a system 230. Hereinafter, an example in which the medical system 1 is a system utilizing a surgical rigid scope will be described. Basically, a treatment tool is inserted to an abdominal cavity and the like as a device separate from the rigid scope. However, the detection technique of the present embodiment is applicable to a system utilizing a flexible scope for a digestive tract. In such case, the treatment tool protrudes from a tip of the flexible scope through a treatment tool channel of the flexible scope.

The endoscope system 200 is a system for capturing an image of inside of a body cavity. The endoscope system 200 includes an endoscope and a main body device.

The endoscope is a rigid scope which is inserted in the body cavity and images inside of the body cavity. The endoscope includes an insertion section to be inserted into the body cavity, an operation section connected to a base end of the insertion section, a universal cord connected to a base end of the operation section, and a connector section connected to a base end of the universal cord. A distal end of the insertion section is provided with an imaging device for imaging inside of the body cavity, and an illumination optical system for illuminating inside of the body cavity. The imaging device includes an objective optical system and an image sensor which captures an image of an object formed by the objective optical system. The connector section detachably connects a transmission cable to the main body device. An image captured by the endoscope is to be referred to as a captured image or an endoscopic image.

The main body device includes a processing device which performs control of the endoscope, image processing of an endoscopic image, and display processing of an endoscopic image, and alight source device which generates and controls illumination light. The processing device includes a processor such as a CPU, and performs image processing of image signals transmitted from the endoscope to generate an endoscopic image and outputs the endoscopic image to a display and the controller 100. The illumination light emitted by the light source device is guided to an illumination optical system of the endoscope by a light guide and emitted from the illumination optical system into the body cavity.

The equipment or the system 230 is an equipment or a system which operates in response to output from the controller 100. The equipment or the system 230 is, for example, a display that displays a contact state or support information based on the contact state. This display may be separately provided from the display of the endoscope system 200 or may be the display of the endoscope system 200. Alternatively, the equipment or the system 230 may be an energy device and a generator which drives the energy device. The energy device is a device which outputs energy of high frequency power or ultrasound, etc. from a distal end section thereof to perform treatment, such as coagulation, sealing, hemostasis, incision, division, or dissection, to a tissue in contact with the distal end section. The energy device is a monopolar device which outputs electrical energy from a single end effector, a bipolar device which applies electrical energy between jaws, an ultrasonic device which outputs ultrasonic energy, a combined device which uses ultrasonic energy and electrical energy together, or the like. The generator controls energy output based on the contact state or the support information based on the contact state from the controller 100.

Note that the medical system 1 may further include a treatment tool which is a non-energy device such as forceps or a spatula. The treatment tool as a target of contact detection may be any of an energy device and a non-energy device.

The controller 100 includes a processor 110, a memory 120, an I/O device 180, and an I/O device 190. Provided herein is an example in which the controller 100 is a separate device from the main body device of the endoscope system 200. The controller 100 may be an information processing device such as a personal computer, or a cloud system configured with a plurality of information processing devices connected through network. However, a function of the controller 100 may be configured to be built in the main body device of the endoscope system 200.

The I/O device 180 receives the endoscopic image from the endoscope system 200. The I/O device 180 is a cable connector for connection to the main body device of the endoscope system 200, or a communication circuit which performs communication processing with the main body device.

The I/O device 190 outputs, to the equipment or the system 230, information about the contact state or the support information based on the contact state that is output by the processor 110. The I/O device 190 is a cable connector for connection to the equipment or the system 230 or a communication circuit which performs communication processing with the equipment or the system 230.

The processor 110 includes hardware. The processor 110 is, for example, a CPU (Central Processing Unit), a GPU (Graphics Processing Unit), a microcomputer, a DSP (Digital Signal Processor), or the like. Alternatively, the processor 110 may be an ASIC (Application Specific Integrated Circuit), a FPGA (Field Programmable Gate Array), or the like. The processor 110 may be configured with one or more of a CPU, GPU, microcomputer, DSP, ASIC, FPGA, and the like. The memory 120 is, for example, a semiconductor memory that is a volatile memory or a non-volatile memory. Alternatively, the memory 120 may be a magnetic storage device such as a hard disk device, or an optical storage device such as an optical disk device.

The memory 120 stores a program 121 that describes process contents of a treatment tool detection section 130 and a contact detection section 140. The processor 110 executes the program 121 to perform processing of the treatment tool detection section 130 and the contact detection section 140. For example, the program 121 includes a program module that describes processing of each section and the processor 110 executes the program module to perform the processing of each section.

The program 121 includes a trained model 122 obtained through machine learning. The trained model is, for example, a neural network trained by deep learning. In this case, the trained model includes a program that describes algorithm of the neural network, a weight parameter between nodes of the neural network, and the like. The neural network includes an input layer to which input data is input, an intermediate layer that performs computation processing to the data input by the input layer, and an output layer that outputs an inference result based on a computation result output from the intermediate layer. In a training phase, a training system configured with an information processing device or a cloud system executes machine learning processing. The training system includes a processor and a memory that stores a model and training data. The processor uses the training data to train the model, thereby generating the trained model 122. This trained model 122 is stored in the memory 120 of the controller 100. Note that the controller 100 may also serve as the training system.

Note that a non-transitory information storage medium, which is a computer readable medium, may store the program 121. The information storage medium is, for example, an optical disk, a memory card, a hard disk drive, a semiconductor memory, or the like. The semiconductor memory is, for example, an ROM or a non-volatile memory. The processor 110 loads the program 121 stored in the information storage medium into the memory 120 and performs various processes based on the program 121.

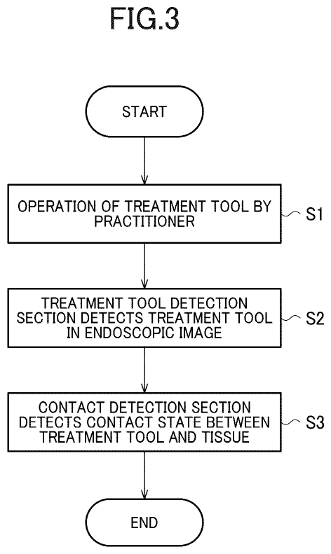

FIG. 3 is an example flow of processing performed by the medical system. In the step S1, a practitioner operates the treatment tool. The endoscope system 200 captures an endoscopic image in which the treatment tool and a treatment target are captured. The treatment target refers to a tissue to be treated by the treatment tool. The processor 110 acquires the endoscopic image from the endoscope system 200.

In the step S2, the treatment tool detection section 130 detects the treatment tool from the endoscopic image. One example of the detection technique is detection or segmentation by AI.

In the step S3, the contact detection section 140 detects a contact state between the treatment tool detected in the step S2 and the tissue from the endoscopic image. The image used for detection may be all or a part of the endoscopic image. For example, the contact detection section 140 may set a region of interest which includes the treatment tool and a surrounding tissue in the endoscopic image based on a detection result by the detection or segmentation, and detect the contact state from an image of the region of interest.

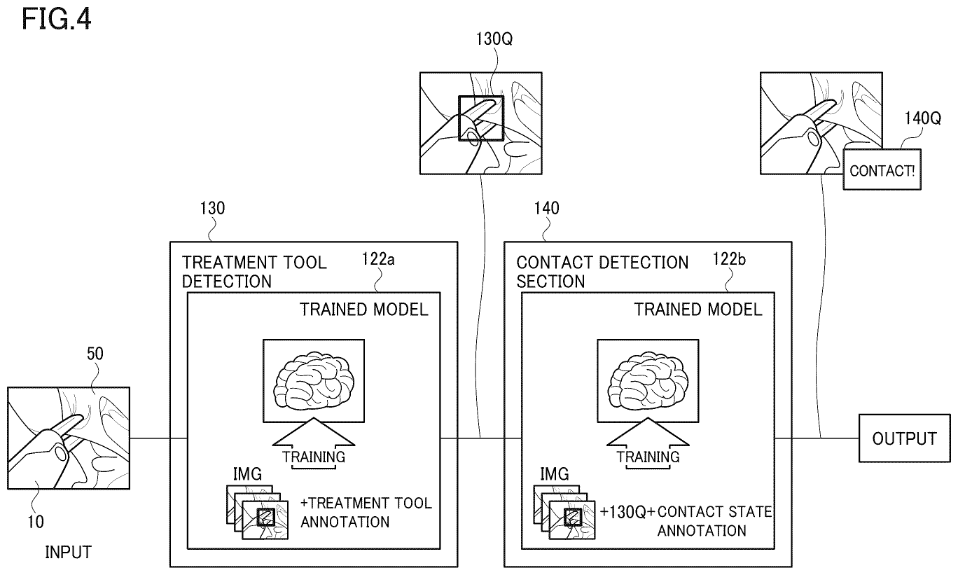

FIG. 4 is a first example configuration of the treatment tool detection section and the contact detection section. FIG. 4 shows processes in a training phase and an inference phase. As described above, the process in the training phase is performed by the training system.

First, the inference phase will be described. The trained model 122 includes a trained model 122a for detection of a treatment tool and a trained model 122b for contact detection. The endoscope system 200 captures an endoscopic image in which a treatment tool 10 and a tissue 50 are captured.

The treatment tool detection section 130 inputs the endoscopic image to the trained model 122a. The trained model 122a detects a position or a region of the treatment tool 10 from the endoscopic image and outputs a detection result 130Q thereof. An example of the detection result 130Q is a bounding box to be detected by detection or a region to be detected by segmentation.

The contact detection section 140 inputs the endoscopic image and the detection result 130Q to the trained model 122b, or inputs the image of the region of interest of the endoscopic image, which is set based on the detection result 130Q, to the trained model 122b. The trained model 122b detects the contact state between the treatment tool 10 and the tissue 50 from the input data, and outputs a detection result 140Q thereof.

The processor 110 performs output using the detection result 140Q. The processor 110 may output the contact state between the treatment tool 10 and the tissue 50 obtained as the detection result 140Q, or output information utilizing the contact state. An example of information output utilizing the contact state will be described.

In a first example, the processor 110 uses the detection result 140Q of the contact state as a trigger of information recognition or information presentation. When contact between the treatment tool 10 and the tissue 50 is detected, the processor 110 determines a tissue type or a tissue condition in a contact area, and presents the information to a practitioner by displaying on a monitor and the like. This enables presentation of the support information at appropriate timing to a non-expert practitioner and the like. Alternatively, when contact between the treatment tool 10 and the tissue 50 is detected, the processor 110 determines an output setting of the energy device according to the tissue type or the tissue condition in the contact area, and recommends the output setting to the practitioner by displaying on a monitor and the like. This allows the support information to be provided at appropriate timing rather than constantly displayed. As described in FIG. 1, when acceptance is input by the practitioner, the processor 110 may instruct output with the recommended setting to the generator, and the generator may perform output from the energy device with the recommended setting.

In a second example, the processor 110 uses the detection result 140Q of the contact state to control the energy device. When contact between the treatment tool 10 and the tissue 50 is detected, the processor 110 may transmit an instruction for releasing a safety lock of the energy device to the generator. In other words, the safety lock is engaged while no contact between the treatment tool 10 and the tissue 50 is being detected, so that no energy output is performed even if output operation of the energy device is performed.

Next, the training phase will be described. The training system trains a model to detect the position or the region of the treatment tool from a training endoscopic image IMG, thereby generating the trained model 122a. The training endoscopic image IMG may be an image captured by an endoscope system which is different from the endoscope system 200 used for the inference phase. The training data corresponds to a plurality of endoscopic images IMG and an annotation indicating the position or the region of the treatment tool in each of the images. The annotation refers to a bounding box indicating the position of the end effector of the treatment tool in the detection, or a region occupied by the end effector of the treatment tool in the segmentation.

The training system trains a model to detect the contact state between the treatment tool and the tissue from input data, thereby generating the trained model 122b. The input data corresponds to the endoscopic image IMG and the annotation indicating the position or the region of the treatment tool. Alternatively, the input data is the image of the region of interest of the endoscopic image IMG, which is set based on the annotation. The training data corresponds to a plurality of endoscopic images IMG, the annotation indicating the position or the region of the treatment tool in each of the images, and a label indicating the contact state between the treatment tool and the tissue in each image. Although the label indicating the contact state herein is assumed to be mainly for a state of contact, non-contact, grasp, or non-grasp, labels of various contact states as described above may be used.

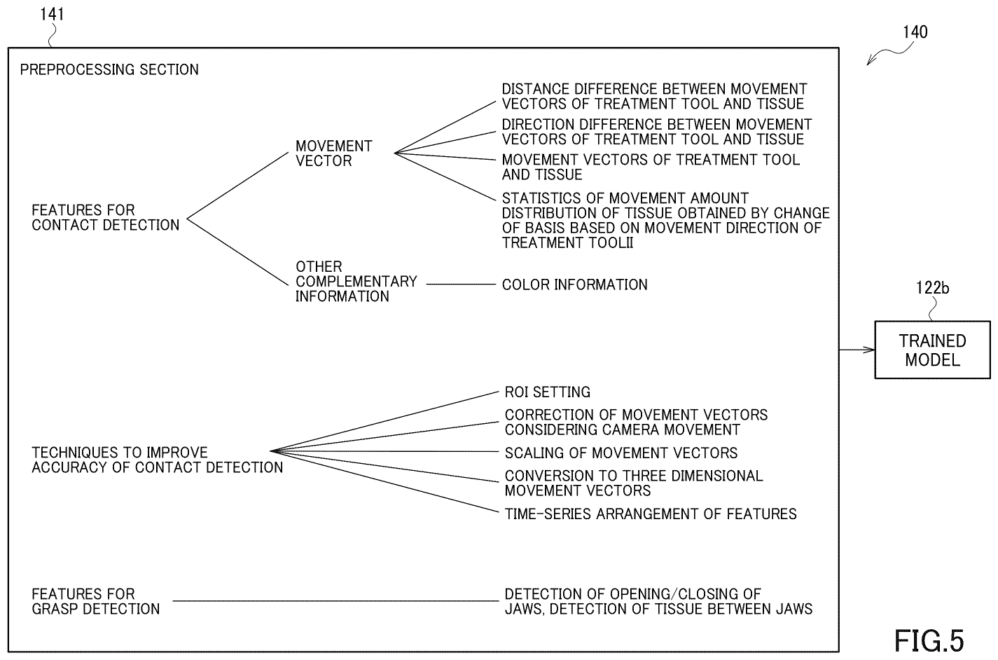

FIG. 5 is a second example configuration of the contact detection section. The contact detection section 140 performs preprocessing by a preprocessing section 141 and contact detection using output by the preprocessing and the trained model 122b. A program realizing the preprocessing section 141 may be a rule-based program or a trained model that is trained through machine learning.

Information to be acquired in the preprocessing is shown in the block of the preprocessing section 141. While the information is hierarchized according to the nature thereof, the information acquired by the preprocessing is “distance difference between treatment tool and tissue in movement direction”, “direction difference between movement vectors of treatment tool and tissue”, . . . , “detection of opening/closing of jaws, detection of tissue between jaws” shown in the lowest level. The preprocessing section 141 acquires these one or more pieces of information and inputs to the trained model 122b. The trained model 122b detects, from the input one or more pieces of information, the contact state between the treatment tool and the tissue. Among the information shown in the block of the preprocessing section 141, the information shown under the level of “other complementary information” and “techniques to improve accuracy of contact detection” is used in combination with the information shown under the level of “movement vector” and “features for grasp detection”. In the training phase of the trained model 122b, the training data includes one or more pieces of information as input data, and a label of the contact state corresponding to the input data.

Hereinafter, the details of the information acquired by the preprocessing will be described. However, “detection of opening/closing of jaws, detection of tissue between jaws” will be described later in the second embodiment.

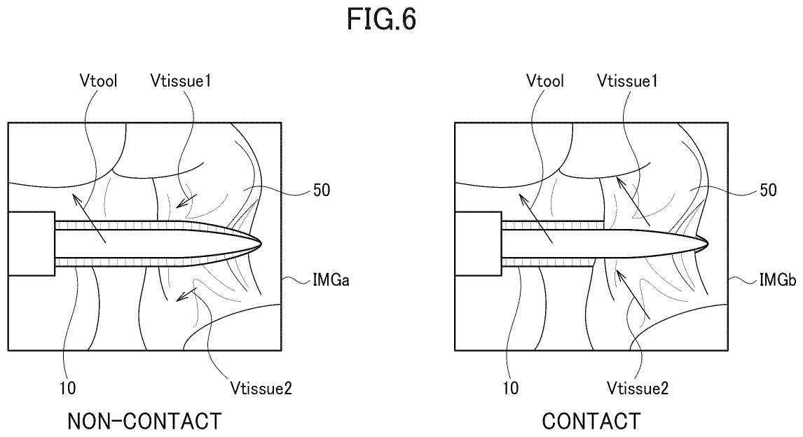

FIG. 6 is a diagram illustrating movement vectors of the treatment tool and the tissue.

The preprocessing section 141 uses a technique of optical flow or image registration to obtain a movement vector from the endoscopic image. A movement vector at each pixel, a movement vector at each point of a grid, a movement vector at a feature point of an image, or representative movement vectors of the treatment tool and the tissue may be obtained. The representative movement vector of the treatment tool is, for example, an average movement vector in the treatment tool region detected by segmentation and the like. The representative movement vector of the tissue is, for example, an average movement vector in a region around the treatment tool region detected by the segmentation. The average movement vector is not limited to a simple average of movement vectors, and may be calculated by a weighted average. Taking the average movement vector of the treatment tool for example, the average movement vector is calculated by calculating a confidence level for each movement vector in movement vector distribution of the treatment tool region, and weighting based on the confidence level to obtain a weighted average of the movement vector. The same applies to the average movement vector of the tissue.

As illustrated in FIG. 6, suppose that a movement vector Vtool of the treatment tool 10 and movement vectors Vtissue1 and Vtissue2 of the tissue 50 are obtained. In the endoscopic image IMGa at a time of non-contact, the surrounding tissue does not move even if the treatment tool moves, so that the movement of the treatment tool is less relevant to the movement of the surrounding tissue. In other words, at the time of non-contact, correlation between the movement vector Vtool of the treatment tool 10 and the movement vectors Vtissue1 and Vtissue2 of the tissue 50 is small. On the other hand, in the endoscopic image IMGb at a time of contact, when the treatment tool moves, the surrounding tissue makes a similar movement, so that the movement of the treatment tool is highly relevant to the movement of the surrounding tissue. In other words, at the time of contact, the correlation between the movement vector Vtool of the treatment tool 10 and the movement vectors Vtissue1 and Vtissue2 of the tissue 50 is large. According to these, it is possible to detect contact using the movement vectors.

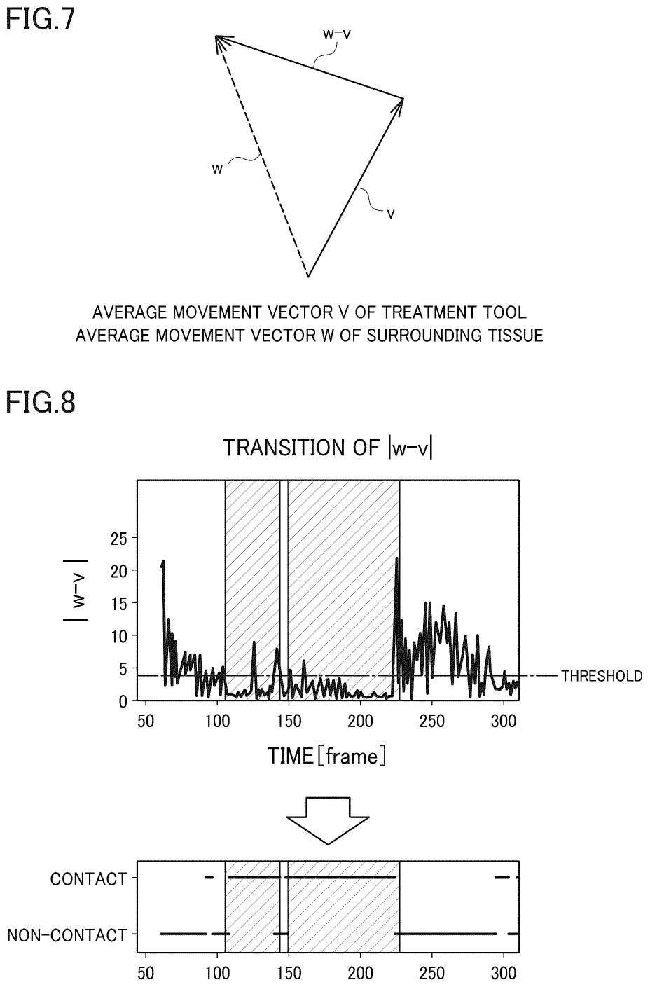

FIGS. 7 and 8 are diagrams illustrating the “distance difference between treatment tool and tissue in movement direction” obtained by the preprocessing section.

As illustrated in FIG. 7, the preprocessing section 141 calculates an average movement vector v of the treatment tool and an average movement vector w of the surrounding tissue around the treatment tool, and calculates Euclidean distance |w−v| between these average movement vectors. The smaller the Euclidean distance |w−v|, the greater the similarity between the movement vectors y and w, and thus, the treatment tool is considered to be in contact with the tissue. In other words, the trained model 122b increases a probability of a determination result that the treatment tool is in contact with the tissue as the Euclidean distance |w−v| decreases.

As illustrated in FIG. 8, the preprocessing section 141 obtains time-series transition of the Euclidean distance |w−v| and inputs it to the trained model 122b. The trained model 122b makes determination of non-contact in a time period with the Euclidean distance |w−v| larger than a threshold, and makes determination of contact in a time period with the Euclidean distance |w−v| smaller than the threshold. However, since the trained model 122b makes determination through a network obtained by training, it is not a simple result of threshold determination.

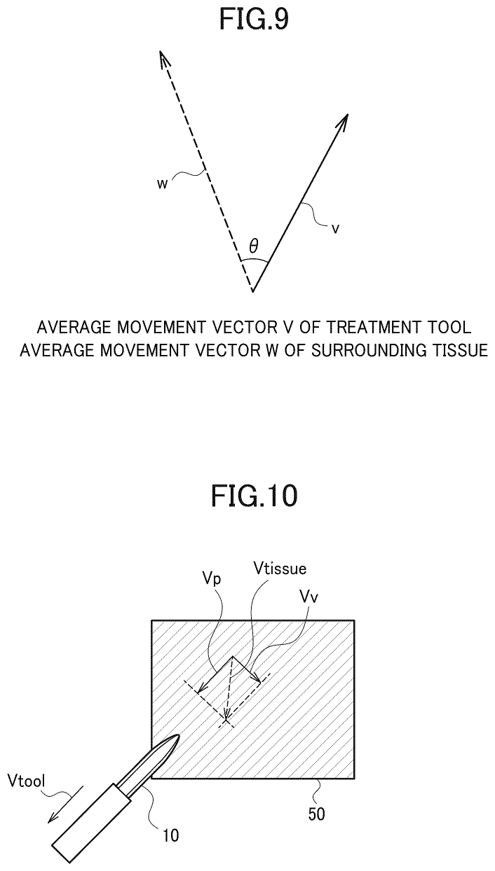

FIG. 9 is a diagram illustrating the “direction difference between treatment tool and tissue in movement direction” obtained by the preprocessing section.

The preprocessing section 141 calculates direction difference between the average movement vector v of the treatment tool and the average movement vector w of the surrounding tissue around the treatment tool. An example of the direction difference is cosine similarity. When an angle formed by the average movement vector v of the treatment tool and the average movement vector w of the surrounding tissue around the treatment tool is θ, the cosine similarity is cos θ. The closer the cosine similarity cos θ is to 1, the greater the similarity between the movement vectors v and w is, and thus, the treatment tool is considered to be in contact with the tissue. In other words, the trained model 122b increases the probability of the determination result that the treatment tool is in contact with the tissue as the cosine similarity cos θ approaches to 1.

The “movement vectors of treatment tool and tissue” obtained by the preprocessing section will be described.

The trained model 122b utilizes each of the movement vectors of the treatment tool and the surrounding tissue as features for contact detection. The movement vectors of the treatment tool and the surrounding tissue themselves can be useful features for contact detection.

For example, if an absolute value of the movement vector is small, it is considered that the influence of noise is large and reliability of contact recognition is low. For example, when the treatment tool or the treatment tool is actually moving very little, the effect of camera shake will be relatively large and thus, it is considered to be difficult to appropriately determine the contact state based on the Euclidean distance or the cosine similarity described above. Therefore, when the absolute value of the movement vector of the treatment tool or the surrounding tissue is small, the trained model 122b determines the reliability of contact recognition as low. For example, when the above absolute value is small, the trained model 122b may not output a recognition result of the contact state or output a result “recognition impossible”.

Alternatively, when the treatment tool is in contact with the tissue, especially when the treatment tool is grasping the tissue, the movement amount of the treatment tool is considered to be small as compared to a case where the treatment tool can freely move. This is because it is considered that camera shake is reduced or positioning is performed. For example, when the absolute value of the movement vector of the treatment tool is small, the trained model 122b may determine the contact state based thereon. As one example, when the absolute value of the movement vector of the treatment tool is small, the trained model 122b may retain the recognition result of the contact state previously obtained.

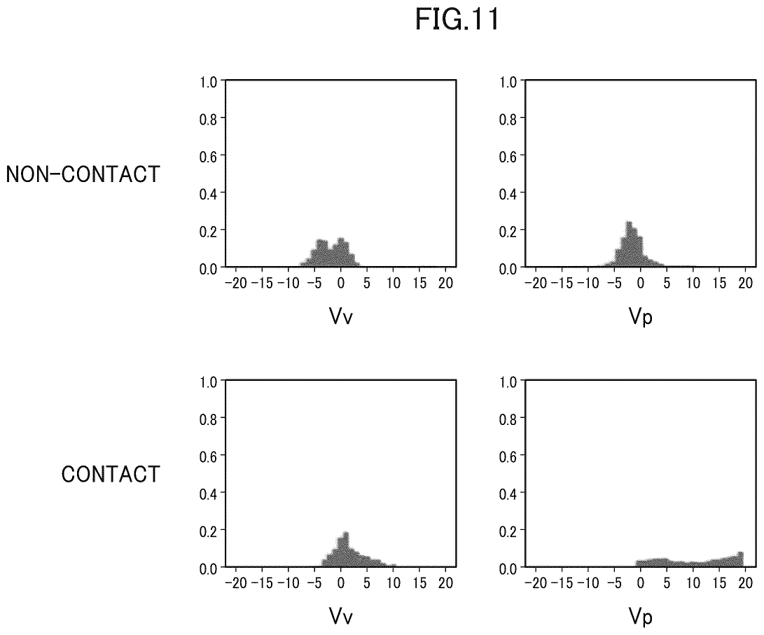

FIGS. 10 and 11 are diagrams illustrating “statistics of movement amount distribution of tissue obtained by change of basis based on movement direction of treatment tool” obtained by the preprocessing section.

As illustrated in FIG. 10, the preprocessing section 141 calculates a movement direction MDtool of the treatment tool 10 and a movement vector Vtissue of the surrounding tissue 50 around the treatment tool. The preprocessing section 141 decomposes the movement vector Vtissue of the surrounding tissue 50 into a parallel component Vp and a vertical component Vv relative to the movement direction MDtool of the treatment tool 10. The movement direction MDtool of the treatment tool 10 is a direction longitudinal to the treatment tool 10 or a direction of the movement vector of the treatment tool 10. The trained model 122b determines the contact state by using the components Vp and Vv.

FIG. 11 shows histograms of the components Vp and Vv obtained at a plurality of positions of the surrounding tissue in one frame. At the time of non-contact, both the histograms of the parallel component Vp and the vertical component Vv are generally symmetrical about “0”. On the other hand, at the time of contact, the histogram of the vertical component Vv is generally symmetrical about “0” while the histogram of the parallel component Vp is clearly asymmetrical about “0”. According to these, it is possible to determine the contact state using the components Vp and Vv. For example, the preprocessing section 141 calculates kurtosis and skewness of each histogram as statistics and inputs the kurtosis and the skewness to the trained model 122b. The kurtosis is a measure of sharpness of a histogram. The skewness is a measure of asymmetry of a histogram.

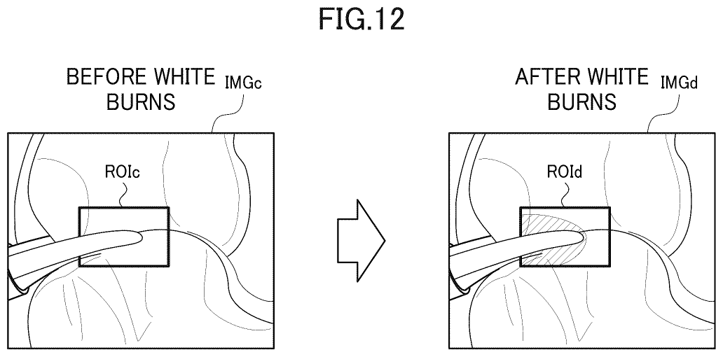

FIG. 12 is a diagram illustrating “color information” acquired by the preprocessing section. Here, the treatment tool is an energy device.

The trained model 122b utilizes color information of the surrounding tissue around the treatment tool as the features for contact detection. By utilizing the color information, change such as white burns or mist of the tissue caused during energy treatment is captured, and such information is used to complement determination of the contact state. Occurrence of white burns or mist means that the energy treatment is going on, i.e. it can be determined as “contact”.

As illustrated in the left figure of FIG. 12, suppose that a region of interest ROIc is set at a distal end of the treatment tool in the endoscopic image IMGc before white burns. In the region of interest ROIc, color of the tissue includes a strong red component. In other words, the red component is sufficiently greater than a blue component, and the red component is sufficiently greater than a green component. As illustrated in the right figure of FIG. 12, suppose that a region of interest ROId is set at the distal end of the treatment tool in the endoscopic image IMGd after white burns. The hatching represents a burned white area. In the region of interest ROId, the color of the tissue is changed to white, such that there is almost no difference between each color component. In other words, the red component is almost the same as the blue and green components. As such, it is possible to determine the white burns by the color components. For example, the preprocessing section 141 obtains an O1 channel of an opposite color space represented by the following formula (1) and inputs it to the trained model 122b.

$$\begin{matrix}[ Formula 1 ] & \\ ( \begin{matrix}O 1 \\ O 2 \\ O 3\end{matrix} ) = ( \begin{matrix}\frac{R - G}{\sqrt{2}} \\ \frac{R + G - 2 B}{\sqrt{6}} \\ \frac{R + G + B}{\sqrt{3}}\end{matrix} ) & ( 1 )\end{matrix}$$

The “ROI setting” acquired by the preprocessing section will be described. ROI stands for Region Of Interest.

The preprocessing section 141 sets the region of interest in the endoscopic image, calculates the features based on the image of the region of interest, and inputs the features to the trained model 122b. The region of interest includes a portion of the treatment tool for which contact detection is desired and the surrounding tissue thereof. The trained model 122b detects the contact state between the treatment tool and the tissue from the input features. The features are the features with the movement vector or the color information described above, or the features for grasp detection described later. The movement vectors and the like of the treatment tool and the surrounding tissue are relevant to each other basically around the contact area. Accordingly, by setting the region of interest, it is possible to improve detection accuracy of the contact state.

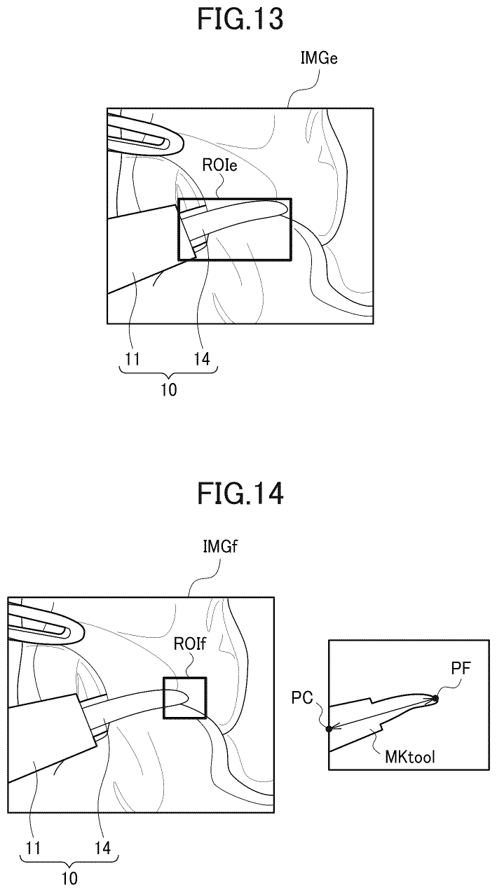

FIG. 13 is a first example of the setting of the region of interest. The treatment tool 10 has a shaft 11 and an end effector 14. The preprocessing section 141 sets a region of interest ROIe around the end effector 14. FIG. 13 shows an example in which the region of interest ROIe includes the entire end effector 14 and a surrounding area thereof. For example, by detection processing using machine learning, the end effector 14 is detected from the endoscopic image IMGe. The preprocessing section 141 may perform the detection processing, or in a case where the treatment tool detection section 130 detects the treatment tool by detection, the preprocessing section 141 may use the detection result by the treatment tool detection section 130. The preprocessing section 141 directly uses the detected region as the region of interest ROIe, or expands or reduces the detected region and uses it as the region of interest ROIe.

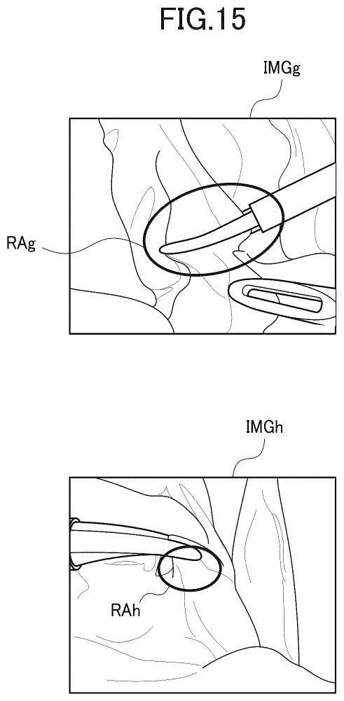

FIG. 14 is a second example of the setting of the region of interest. As illustrated in the left figure, the preprocessing section 141 sets a region of interest ROIf around the distal end section of the end effector 14. For example, by segmentation processing using machine learning, the region of the treatment tool 10 is detected from the endoscopic image IMGf. The preprocessing section 141 may perform the segmentation processing, or in a case where the treatment tool detection section 130 detects the treatment tool by the segmentation, the preprocessing section 141 may use the detection result by the treatment tool detection section 130. The preprocessing section 141 detects the distal end of the end effector 14 from the detected region of the treatment tool 10, and sets the region of interest ROIf on the basis of the distal end. An example of detection of the distal end section is shown in the right figure. A treatment tool mask MKtool is the region of the treatment tool 10 detected by the segmentation. The preprocessing section 141 obtains a midpoint PC of an intersection of the edge of the image and the treatment tool mask MKtool, obtains the furthest point PF from the midpoint PC in the treatment tool mask MKtool, and determines the midpoint PF as the distal end of the end effector 14.

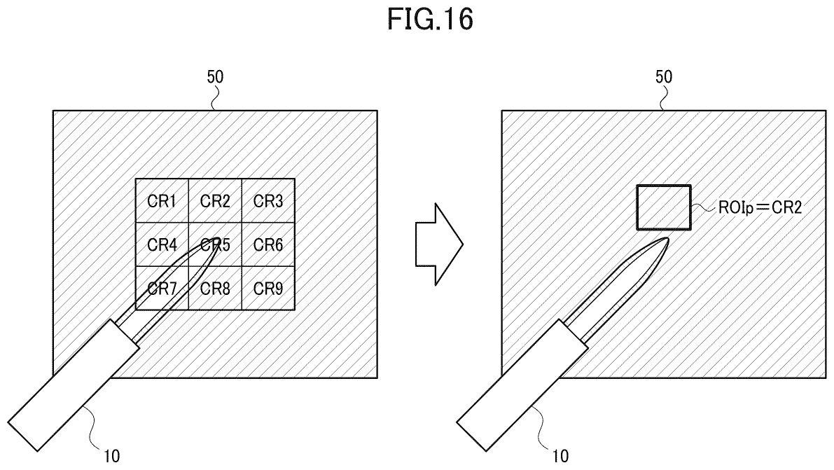

FIGS. 15 and 16 show a third example of the setting of the region of interest.

FIG. 15 shows example images with different optimum positions of the region of interest. The endoscopic image IMGg is an example image when a long length of a tissue is grasped, while the endoscopic image IMGh is an example image when a short length of the tissue is grasped. The movement of the tissue is highly correlated to the movement of the treatment tool in the area grasped by a jaw, which is an effector. Therefore, when the long length of the tissue is grasped, the tissue is grasped by more than half part of the jaw so that a surrounding region RAg thereof is appropriate as the region of interest. On the other hand, when the short length of the tissue is grasped, the tissue is grasped only by a part near the distal end of the jaw so that a region RAh near the distal end of the jaw is appropriate as the region of interest.

As illustrated in FIG. 16, the preprocessing section 141 generates a plurality of region of interest candidates CR1-CR9 around the distal end of the treatment tool 10. For example, the preprocessing section 141 sets a region of a certain size on the basis of the distal end of the treatment tool 10, and divides the region to generate a plurality of region of interest candidates CR1-CR9. Note that the number of the candidates is not limited to 9. The preprocessing section 141 sets one or more of the plurality of region of interest candidates CR1-CR9 as a region of interest ROIp. FIG. 16 shows an example in which the candidate CR2 is set as the region of interest ROIp. The preprocessing section 141 selects, for example, a region of interest candidate having a movement vector close to the movement vector of the treatment tool as the region of interest ROIp. Note that the FIG. 15 shows an example in which the end effector of the treatment tool is a jaw, but the technique in FIG. 16 is applicable to a case where the end effector is not a jaw.

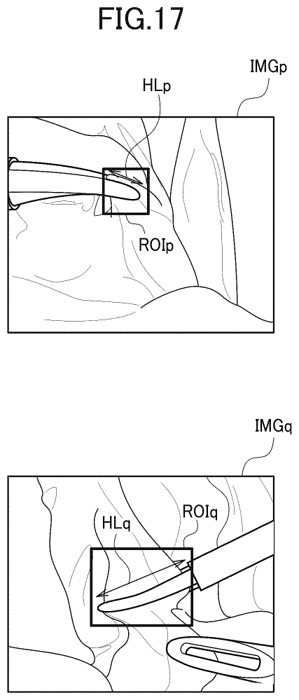

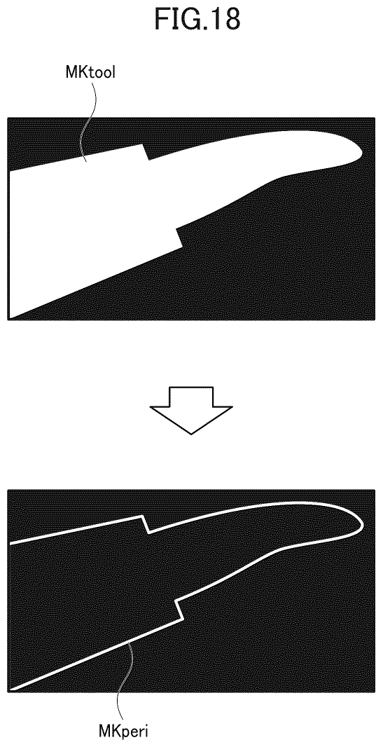

FIGS. 17 and 18 show a fourth example of the setting of the region of interest. Here, the end effector of the treatment tool is a jaw.

The endoscopic image IMGp is an example image when a short length of the tissue is grasped. A part of the jaw grasping the tissue is indicated by HLp. If the region of interest including the entire jaw is set, most of the tissue included in the region of interest is not grasped and thus, movement correlation between the jaw and the surrounding tissue becomes small. The endoscopic image IMGq is an example image when the long length of the tissue is grasped. The part of the jaw grasping the tissue is indicated by HLq. If the region of interest including only the distal end of the jaw is set, the most of the region where the movement correlation between the jaw and the surrounding tissue is large will not be included in the region of interest.

Therefore, the preprocessing section 141 detects the center of the part of the jaw grasping the tissue, and sets the region of interest on the basis of the center. In other words, the preprocessing section 141 detects the center of the parts HLp and HLq of the jaw grasping the tissue from the endoscopic images IMGp and IMGq, and sets the regions of interest ROIp and ROIq on the basis of the center. This enables setting of the region of interest including the region where the movement correlation between the jaw and the surrounding tissue is large, thereby improving the accuracy of contact detection.

FIG. 18 is an example technique of detecting the center of the part of the jaw grasping the tissue. By the segmentation processing, the region of the treatment tool 10 is detected from the endoscopic image IMGf. The preprocessing section 141 may perform the segmentation processing, or in a case where the treatment tool detection section 130 detects the treatment tool by the segmentation, the preprocessing section 141 may use the detection result by the treatment tool detection section 130. The detected region is to be the treatment tool mask MKtool. The preprocessing section 141 performs dilation processing to the treatment tool mask MKtool and obtains difference by subtracting the treatment tool mask MKtool before the dilation from the mask after the dilation. Thereby, a peripheral region mask MKperi along the outline of the treatment tool is obtained.

The preprocessing section 141 performs edge extraction to an image of the peripheral region mask MKperi of the endoscopic image, and detects an area with a great edge component as a ridge of the grasped tissue. The preprocessing section 141 uses the technique described in FIG. 14 and the like to detect the distal end of the treatment tool. The preprocessing section 141 sets a midpoint of a line connecting the ridge of the grasped tissue and the distal end of the treatment tool as a reference point for setting of the region of interest. When the ridge is not detected by the edge extraction, the preprocessing section 141 sets the region of interest using the first example of the setting of the region of interest and the like.

The “correction of movement vectors considering camera movement” performed by the preprocessing section will be described. Since movement of a camera is apparent amount of movement amount, it affects the movement vectors of the treatment tool and the tissue. Accordingly, cancellation thereof is expected to improve the accuracy of contact detection.

The preprocessing section 141 detects the movement amount of the camera and uses the movement amount of the camera to cancel the effect of the movement of the camera from the movement vectors of the treatment tool and the tissue. For example, the preprocessing section 141 subtracts the movement vector of the camera from the movement vectors of the treatment tool and the tissue. The preprocessing section 141 uses the movement vectors after the cancellation to calculate the features such as distance difference. Various known arts can be employed as a technique for obtaining the movement amount of the camera. For instance, the below two examples can be employed. In a first example, a motion sensor or a position sensor by means of an optical sensor, a magnetic sensor, or an inertial sensor is installed on the camera of the endoscope. The preprocessing section 141 detects the movement amount of the camera based on sensor output thereof. In a second example, the preprocessing section 141 estimates the movement amount of the camera by image processing to the endoscopic image. For example, the preprocessing section 141 may detect a global movement vector which represents the movement of the entire image, and estimate the movement amount of the camera based on the global movement vector.

The “scaling of movement vectors” performed by the preprocessing section will be described. A size of a movement vector on an image is affected by both a size of movement in real space and a distance between a camera and an object. Accordingly, by performing scaling using depth distance information or information about a treatment tool width and the like, influence of the distance between the camera and the object can be eliminated and the accuracy of contact detection can be expected to improve.

The preprocessing section 141 detects the distance between the camera and the object. For example, the endoscope has a 3D camera and the preprocessing section 141 uses stereo vision by the 3D camera to detect the distance to the object. Alternatively, the preprocessing section 141 uses known length such as a shaft width of the treatment tool to detect the distance from the endoscopic image to the object. Taking the shaft width for example, the preprocessing section 141 estimates the distance to the object based on the ratio of the shaft width captured in the endoscopic image to the known shaft width. The preprocessing section 141 scales the movement vectors of the treatment tool and the tissue to movement vectors at a reference distance using the detected distance. The preprocessing section 141 uses the movement vectors after scaling to calculate the features such as distance difference.

The “conversion to three-dimensional movement vectors” performed by the preprocessing section will be described.

The preprocessing section 141 uses depth information to obtain the movement vectors of the treatment tool and the tissue as three-dimensional vectors, and uses the three-dimensional movement vectors to calculate the features such as distance difference. The preprocessing section 141 combines, for example, two-dimensional optical flow and the depth information to calculate the three-dimensional movement vectors. This method is called Scene Flow. For example, the endoscope has a 3D camera and the preprocessing section 141 uses stereo vision by the 3D camera to detect the movement amount in a depth direction.

The following advantages can be obtained by using the three-dimensional movement vectors. That is, movement in real space is three-dimensional while image information is projected on a two-dimensional plane, so that the movement information in the depth direction is lost. By using the three-dimensional movement vectors, it is possible to capture the movement in a normal direction of the image with high sensitivity. This improves the confidence level of the movement vectors and thus the accuracy of contact detection.

The “time-series arrangement of features” performed by the preprocessing section will be described.

The preprocessing section 141 inputs time-series features obtained from a plurality of frames in chronological order to the trained model 122b. The trained model 122b detects the contact state between the treatment tool and the tissue from the time-series features. By utilizing the features in a plurality of recent frames upon contact detection, the accuracy of contact detection can be expected to improve.

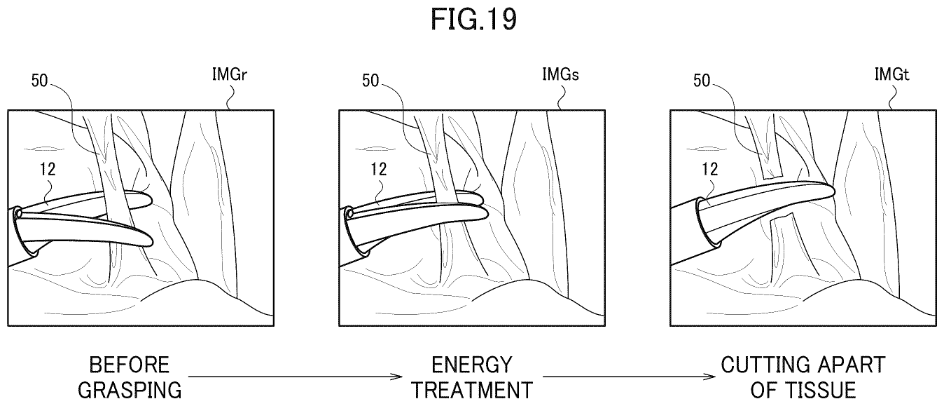

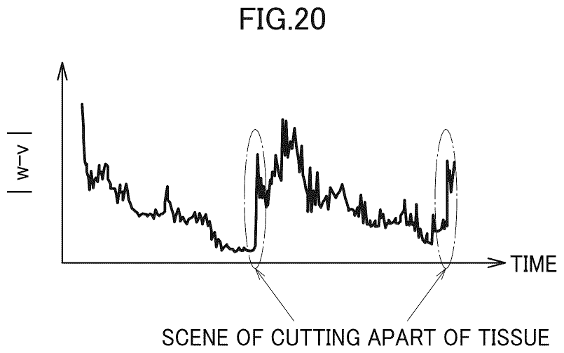

FIGS. 19 and 20 are diagrams illustrating detection of cutting apart of a tissue. Here, the treatment tool is an energy device and the end effector is a jaw.

The advantage of grasp state determination includes detection of cutting apart of a tissue from an image. The detection of cutting apart of a tissue is expected to be effective in preventing damage to a probe of an ultrasonic device during a procedure, and the like. In a technique of contact detection using movement vectors, detection is easy because the features rapidly changes at a scene of cutting apart. The description will be given below.

As illustrated in FIG. 19, in the endoscopic image IMGr, a jaw 12 does not grasp the tissue 50. In the subsequent endoscopic image IMGs, the jaw 12 is grasping the tissue and energy is applied to the tissue 50 between the jaws. At this point, the tissue grasped between the jaws is connected. In the subsequent endoscopic image IMGt, the tissue 50 between the jaws is cut by energy treatment. As such, a tissue being cut by energy treatment is referred to as cutting apart of a tissue. Note that although FIG. 19 shows an example in which the treatment tool is a bipolar device, it is possible to detect cutting apart by the technique of the present embodiment even when using a hook knife-type monopolar device and the like.

FIG. 20 is an example of time-series change in the Euclidean distance |w−v| between the movement vector of the treatment tool and the movement vector of the tissue. In a state that the tissue 50 is grasped by the jaw 12 as in IMGs in FIG. 19, |w−v| is small. When the tissue 50 is cut apart as in IMGt, the correlation between the movement vectors of the jaw 12 and the tissue 50 becomes small. Therefore, as illustrated by the dashed-dotted line circle in FIG. 20, |w−v| suddenly becomes large at the moment of cutting apart. The contact detection section 140 detects such timing at which |w−v| suddenly becomes large, thereby detecting cutting apart of the tissue.

### 3. Second Embodiment

In the second embodiment, the end effector of the treatment tool is a jaw. Note that the description of the parts similar to those in the first embodiment are omitted. In the second embodiment, for example, the configuration of the medical system 1, the configuration of the controller 100, and the processing performed by the treatment tool detection section 130, etc. are similar to those in the first embodiment.

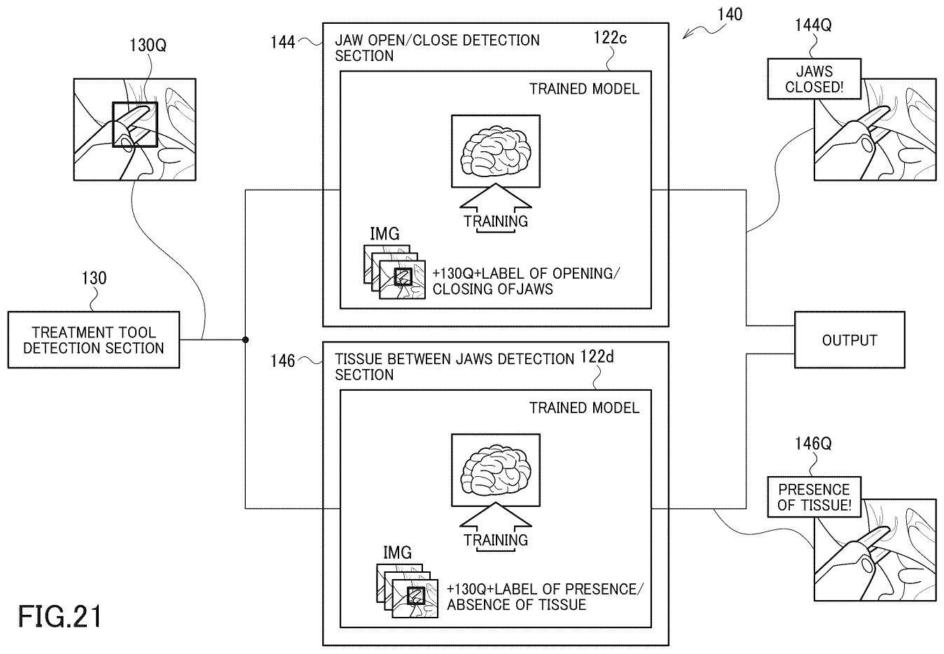

FIG. 21 is a first example configuration of the contact detection section in the second embodiment. The contact detection section 140 includes a jaw open/close detection section 144 and a tissue between jaws detection section 146. FIG. 21 shows the processes in the training phase and the inference phase.

First, the inference phase will be described. The trained model 122 described above with reference to FIG. 2 includes a trained model 122c for detection of opening/closing of jaws and a trained model 122d for detection of a tissue between jaws.

The jaw open/close detection section 144 uses the endoscopic image, the detection result 130Q by the treatment tool detection section 130, and the trained model 122c to detect an open/closed state of the jaws captured in the endoscopic image. The jaw open/close detection section 144 inputs the endoscopic image and the detection result 130Q to the trained model 122c, or inputs the image of the region of interest of the endoscopic image, which is set based on the detection result 130Q, to the trained model 122c. The trained model 122c detects the open/closed state of the jaws from the input data, and outputs a detection result 144Q thereof.

The tissue between jaws detection section 146 uses the endoscopic image, the detection result 130Q by the treatment tool detection section 130, and the trained model 122d to detect presence/absence of a tissue between the jaws captured in the endoscopic image. The tissue between jaws detection section 146 inputs the endoscopic image and the detection result 130Q to the trained model 122d, or inputs the image of the region of interest of the endoscopic image, which is set based on the detection result 130Q, to the trained model 122d. The trained model 122d detects the presence/absence of a tissue between the jaws from the input data and outputs a detection result 146Q thereof. A condition for the presence of a tissue between jaws is, “there is a tissue which covers one of a pair of jaws and does not cover the other jaw”. A tissue covering a jaw means that apart or all of the jaw is hidden by the tissue in an image.

The contact detection section 140 determines the contact state using the detection results 144Q and 146Q. When it is determined that the jaws are closed and there is a tissue between the jaws, the contact detection section 140 determines that the jaws are grasping the tissue, i.e. the treatment tool is in contact with the tissue. A program which determines the contact state using the detection results 144Q and 146Q may be any of a rule-based program or a trained model using machine learning. The processor 110 performs output using the detection results of the contact state. The contents of the output is similar to those in FIG. 4.

As a result, in some embodiments, complex image recognition of detecting the contact state from the image is divided into the jaw open/close detection section 144 and the tissue between jaws detection section 146. This improves recognition accuracy in individual trained models as compared to a case where the contact state is detected by one trained model, so that the recognition accuracy of the contact detection as a whole is expected to improve.

Next, the training phase will be described. The training system trains a model to detect, from input data, the open/closed state of the jaws, thereby generating the trained model 122c. The input data corresponds to the endoscopic image IMG and the annotation indicating the position or the region of the treatment tool. Alternatively, the input data is the image of the region of interest of the endoscopic image IMG, which is set based on the annotation. The training data corresponds to a plurality of endoscopic images IMG, the annotation indicating the position or the region of the treatment tool in each of the images, and a label indicating the open/closed state of the jaws in each image.

The training system trains a model to detect, from input data, the presence/absence of a tissue between the jaws, thereby generating the trained model 122d. The input data corresponds to the endoscopic image IMG and the annotation indicating the position or the region of the treatment tool. Alternatively, the input data is the image of region of interest of the endoscopic image IMG, which is set based on the annotation. The training data corresponds to a plurality of endoscopic images IMG, the annotation indicating the position or the region of the treatment tool in each of the images, and a label indicating the presence/absence of a tissue between the jaws in each image. The label indicating the presence/absence of a tissue between the jaws is attached based on the above-mentioned condition for the presence of a tissue between jaws. By performing machine learning with such training data, an image in which “there is a tissue which covers one of a pair of jaws and does not cover the other jaw” is to be determined as the presence of a tissue between jaws.

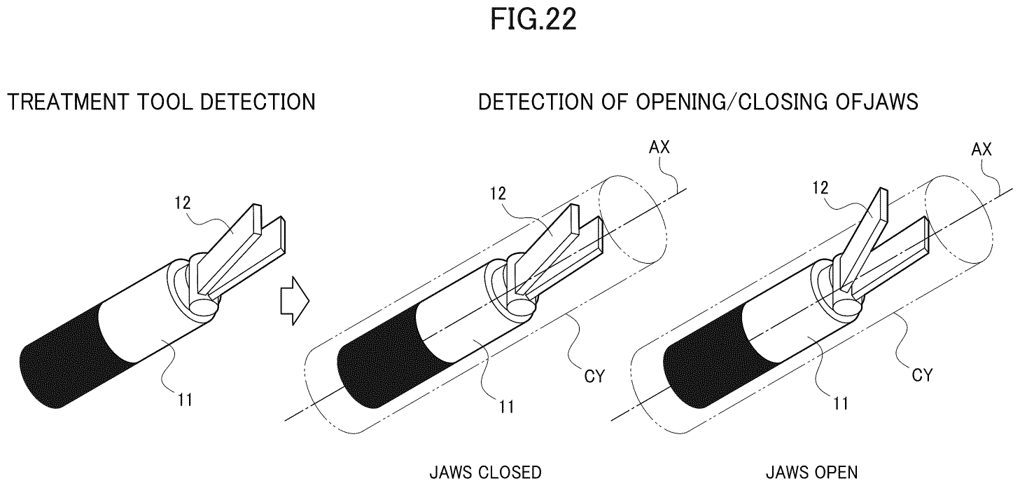

FIG. 22 is a diagram illustrating a second example configuration of the jaw open/close detection section. In this example, the jaw open/close detection section 144 determines the open/closed state of the jaws by a rule-based program using three-dimensional information.

The treatment tool detection section 130 acquires, from the endoscopic image, three-dimensional shapes of the shaft 11 and the jaw 12. The treatment tool detection section 130 inputs, for example, a stereo image obtained by a 3D camera to the trained model 122a, and the trained model 122a recognizes, from the input stereo image, the three-dimensional shapes of the shaft 11 and the jaw 12.

The jaw open/close detection section 144 calculates a shaft axis AX from the three-dimensional shape of the shaft 11, and defines a cylinder CY with a specified radius about the shaft axis AX. The jaw open/close detection section 144 determines whether or not the jaw 12 is within the cylinder CY. The jaw open/close detection section 144 determines that the jaw is closed when the jaw 12 is within the cylinder CY, and determines that the jaw is open when at least a part of the jaw 12 is outside the cylinder CY.

As a result, in some embodiments, opening/closing of the jaws is determined using a rule-based program, so that the basis for determination of opening/closing of the jaws is clear as compared to a case using machine learning.

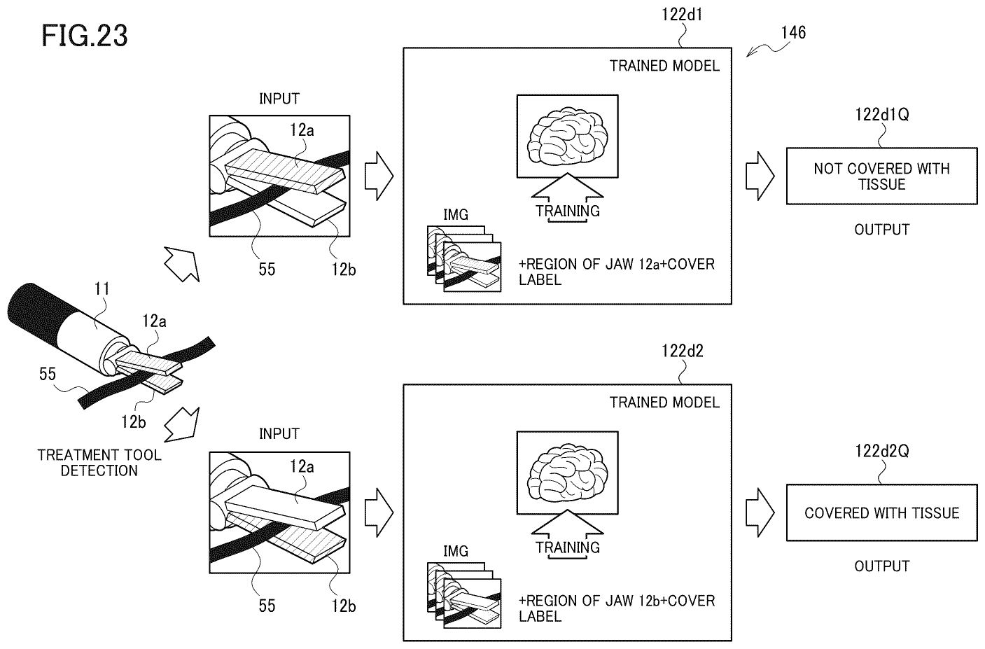

FIG. 23 is a second example configuration of the tissue between jaws detection section. As described above, the condition for the presence of a tissue between jaws is, “there is a tissue which covers one of a pair of jaws and does not cover the other jaw”. In this example, the tissue between jaws detection section 146 determines the presence/absence of a tissue covering each jaw by the trained model. FIG. 23 shows the processes in the training phase and the inference phase.

First, the inference phase will be described. The treatment tool detection section 130 detects a first jaw 12a and a second jaw 12b from the endoscopic image. One of a pair of jaws connected to the shaft 11 is the first jaw 12a and the other is the second jaw 12b.

The trained model 122 described above with reference to FIG. 2 includes a trained model 122d1 for detection of covering of the first jaw and a trained model 122d2 for detection of covering of the second jaw. A determination target of the trained model 122d1 is the first jaw 12a and a determination target of the trained model 122d2 is the second jaw 12b.

The tissue between jaws detection section 146 inputs the endoscopic image and a region of the first jaw 12a detected by the treatment tool detection section 130 to the trained model 122d1. The trained model 122d1 detects, from the input data, whether or not the first jaw 12a is covered with the tissue 55, and outputs a detection result 122d1Q thereof.

The tissue between jaws detection section 146 inputs the endoscopic image and a region of the second jaw 12b detected by the treatment tool detection section 130 to the trained model 122d2. The trained model 122d2 determines, from the input data, whether or not the second jaw 12b is covered with the tissue 55, and outputs a detection result 122d2Q thereof.

The tissue between jaws detection section 146 determines that there is a tissue between the jaws when one of the detection results 122d1Q and 122d2Q is “not covered with the tissue” and the other is “covered with the tissue”. The tissue between jaws detection section 146 determines that there is no tissue between the jaws in a case of other results.

Next, the training phase will be described. The training system trains a model to detect, from input data, whether or not the first jaw 12a is covered with the tissue 55, thereby generating the trained model 122d1. The input data corresponds to the endoscopic image IMG and an annotation indicating the region of the first jaw 12a. The training data corresponds to a plurality of endoscopic images IMG, the annotation indicating the region of the first jaw 12a in each of the images, and a cover label indicating whether or not the first jaw 12a in each image is covered with the tissue 55.

The training system trains a model to detect, from input data, whether or not the second jaw 12b is covered with the tissue 55, thereby generating the trained model 122d2. The input data corresponds to the endoscopic image IMG and an annotation indicating the region of the second jaw 12b. The training data corresponds to a plurality of endoscopic images IMG, the annotation indicating the region of the second jaw 12b in each of the images, and a cover label indicating whether or not the second jaw 12b in each image is covered with the tissue 55.

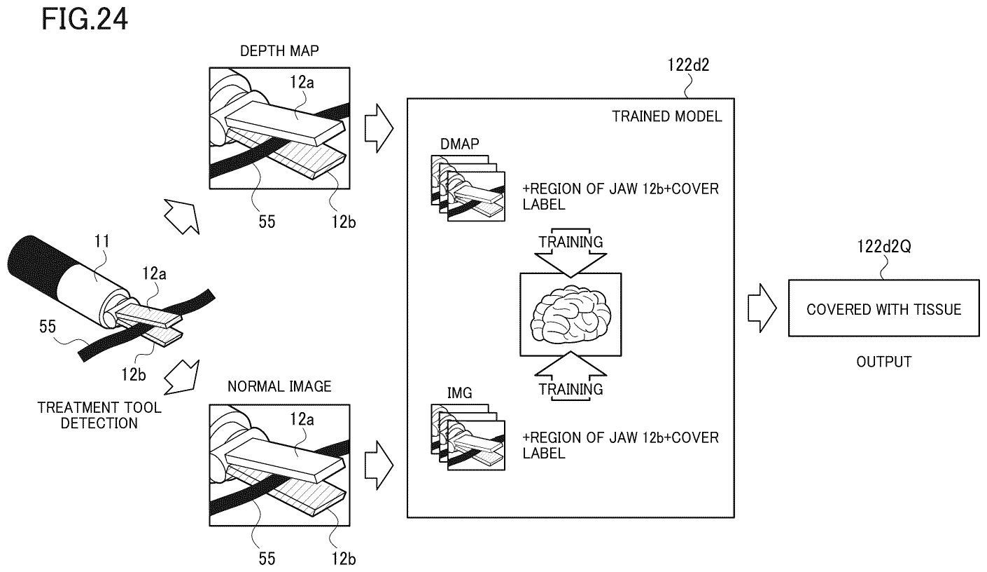

FIG. 24 is a third example configuration of the tissue between jaws detection section. Though FIG. 24 only shows the trained model 122d2 for detection of covering of the second jaw, the trained model 122d1 for detection of covering of the first jaw also performs similar detection and learning targeted at the first jaw 12a. FIG. 24 shows the processes in the training phase and the inference phase.

First, the inference phase will be described. The treatment tool detection section 130 acquires a two-dimensional normal image and a three-dimensional image for the endoscopic image. The three-dimensional image is, for example, a depth map obtained by stereo vision. The treatment tool detection section 130 detects the first jaw 12a and the second jaw 12b from each of the normal image and the three-dimensional image.

The tissue between jaws detection section 146 inputs the three-dimensional image, the region of the second jaw 12b detected from the three-dimensional image, the normal image, and the region of the second jaw 12b detected from the normal image to the trained model 122d2. The trained model 122d2 detects, from the input data, whether or not the second jaw 12b is covered with the tissue 55, and outputs the detection result 122d2Q thereof.

Next, the training phase will be described. The training system trains a model to detect, from input data, whether or not the second jaw 12b is covered with the tissue 55, thereby generating the trained model 122d2. The reference letter “DMAP” in the figure indicates the three-dimensional image and the reference letter “IMG” indicates the normal image. The input data corresponds to the three-dimensional image DMAP, the annotation indicating the region of the second jaw 12b in the three-dimensional image DMAP, the normal image IMG, and the annotation indicating the region of the second jaw 12b in the normal image IMG. The training data corresponds to a plurality of three-dimensional images DMAP, the annotation indicating the region of the second jaw 12b in each three-dimensional image DMAP, a plurality of normal images IMG, the annotation indicating the region of the second jaw 12b in each normal image IMG, and the cover label indicating whether or not the second jaw 12b in each image is covered with the tissue 55. The cover label may be attached to each of the-three dimensional image and the normal image, or one cover label may be attached to a pair of the three-dimensional image and the normal image captured at the same timing.

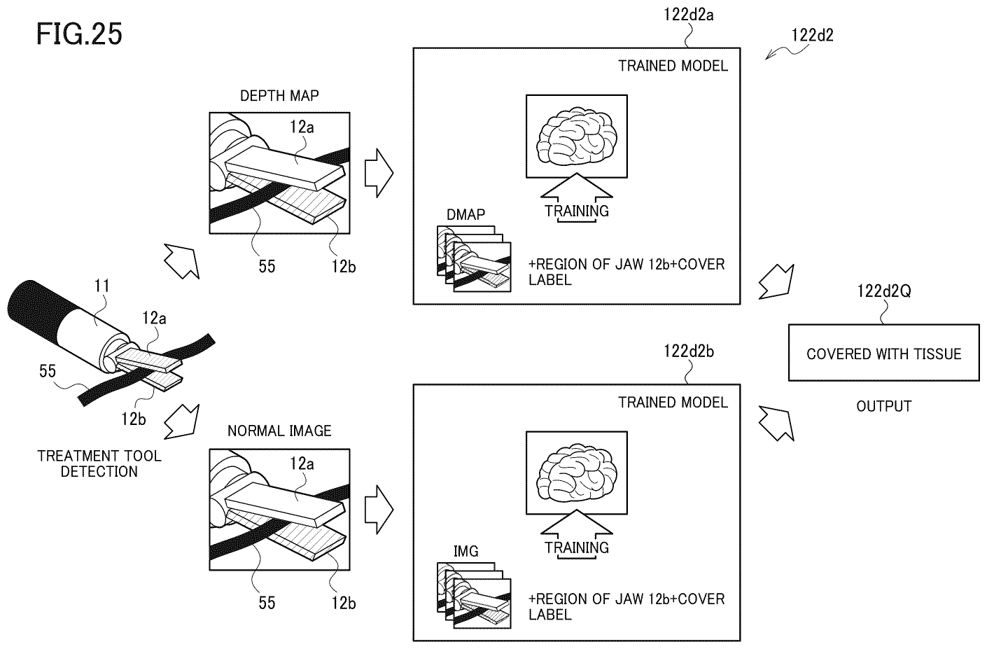

FIG. 25 is a fourth example configuration of the tissue between jaws detection section. Though FIG. 25 only shows the trained model 122d2 for detection of covering of the second jaw, the trained model 122d1 for detection of covering of the first jaw may also perform similar detection and learning targeted at the first jaw 12a. FIG. 25 shows the processes in the training phase and the inference phase.

First, the inference phase will be described. The trained model 122d2 for detection of covering of the second jaw includes a trained model 122d2a for a three-dimensional image and a trained model 122d2b for a normal image.

The tissue between jaws detection section 146 inputs a three-dimensional image and the region of the second jaw 12b detected from the three-dimensional image to the trained model 122d2a. The trained model 122d2a detects, from the input data, whether or not the second jaw 12b is covered with the tissue 55.

The tissue between jaws detection section 146 inputs the normal image and the region of the second jaw 12b detected from the normal image to the trained model 122d2b. The trained model 122d2b detects, from the input data, whether or not the second jaw 12b is covered with the tissue 55.

The tissue between jaws detection section 146 integrates the detection results by the trained model 122d2a and the trained model 122d2b to output the final detection result 122d2Q of whether or not the second jaw 12b is covered with the tissue 55. When the detection result by the trained model 122d2a is the same as the detection result by the trained model 122d2b, the tissue between jaws detection section 146 outputs them as the final detection result 122d2Q. When the detection result by the trained model 122d2a is different from the detection result by the trained model 122d2b, the tissue between jaws detection section 146 outputs the detection result with a higher prediction probability or the detection result selected by preset weighting as the final detection result 122d2Q.

Next, the training phase will be described. The training system trains a model to detect, from input data, whether or not the second jaw 12b is covered with the tissue 55, thereby generating the trained model 122d2a. The input data corresponds to the three-dimensional image DMAP and the annotation indicating the region of the second jaw 12b in the three-dimensional image DMAP. The training data corresponds to a plurality of three-dimensional images DMAP, the annotation indicating the region of the second jaw 12b in each three-dimensional image DMAP, and the cover label indicating whether or not the second jaw 12b in each image is covered with the tissue 55.

The training system trains a model to detect, from input data, whether or not the second jaw 12b is covered with the tissue 55, thereby generating the trained model 122d2b. The input data corresponds to the normal image IMG and the annotation indicating the region of the second jaw 12b in the normal image IMG. The training data corresponds to a plurality of normal images IMG, the annotation indicating the region of the second jaw 12b in each normal image IMG, and the cover label indicating whether or not the second jaw 12b in each image is covered with the tissue 55.

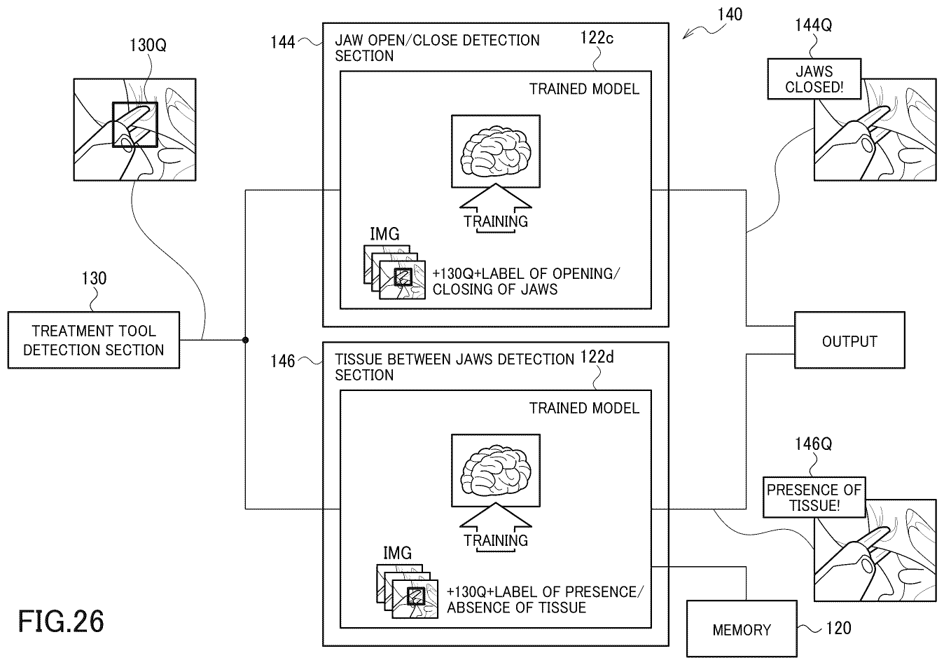

FIG. 26 is a second example configuration of the contact detection section in the second embodiment. The parts different from those in the first example configuration of FIG. 21 will mainly be described.

There is a scene that the presence/absence of a tissue between the jaws cannot be visually recognized after closing the jaws because a pair of jaws overlaps in a depth direction. In such scene, there is a possibility that the tissue between jaws detection section 146 cannot detect the presence/absence of a tissue between the jaws. Hence, a memory function is added to the tissue between jaws detection section 146. The contact detection section 140 retains the recognition result that “there is a tissue between the jaws” by using the memory function, even if the situation is changed after recognizing that “there is a tissue between the jaws” so as not to allow recognition of the presence/absence of the tissue.

Specifically, the memory 120 stores the detection result 140Q by the tissue between jaws detection section 146. For example, the memory 120 retains the detection result 140Q output by the tissue between jaws detection section 146 for a certain period of time from the output timing. The contact detection section 140 uses the detection result 144Q by the jaw open/close detection section 144, the detection result 146Q by the tissue between jaws detection section 146, and the detection result 146Q by the tissue between jaws detection section 146 stored in the memory 120 to detect the contact state between the treatment tool and the tissue. Specifically, when the tissue between jaws detection section 146 cannot determine the presence/absence of the tissue between the jaws, the contact detection section 140 detects the contact state between the treatment tool and the tissue from the detection result 144Q by the jaw open/close detection section 144 and the detection result 146Q by the tissue between jaws detection section 146 stored in the memory 120.

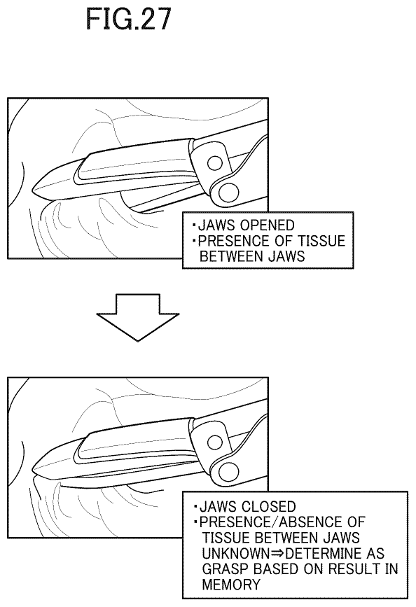

FIG. 27 shows an example of contact detection using the detection result stored in the memory. As illustrated in the upper figure, suppose that at a given first time, the jaw open/close detection section 144 determines that the jaws are open and the tissue between jaws detection section 146 determines that there is a tissue between the jaws. This detection result that “there is a tissue between the jaws” is stored in the memory 120. As illustrated in the lower figure, suppose that at a second time after the first time, the jaw open/close detection section 144 determines that the jaws are closed and the tissue between jaws detection section 146 determines the presence/absence of the tissue between the jaws as unknown. In this case, the contact detection section 140 uses the determination result that “there is a tissue between the jaws” at the first time stored in the memory 120 to determine that the jaws are grasping the tissue, i.e. the treatment tool is in contact with the tissue.

As a result, in some embodiments, if the presence of the tissue between the jaws is recognized when the jaws are open and even the presence/absence of the tissue between the jaws cannot be determined based on the endoscopic image after the jaws are just closed, it is possible to detect the contact state from the result stored in the memory.

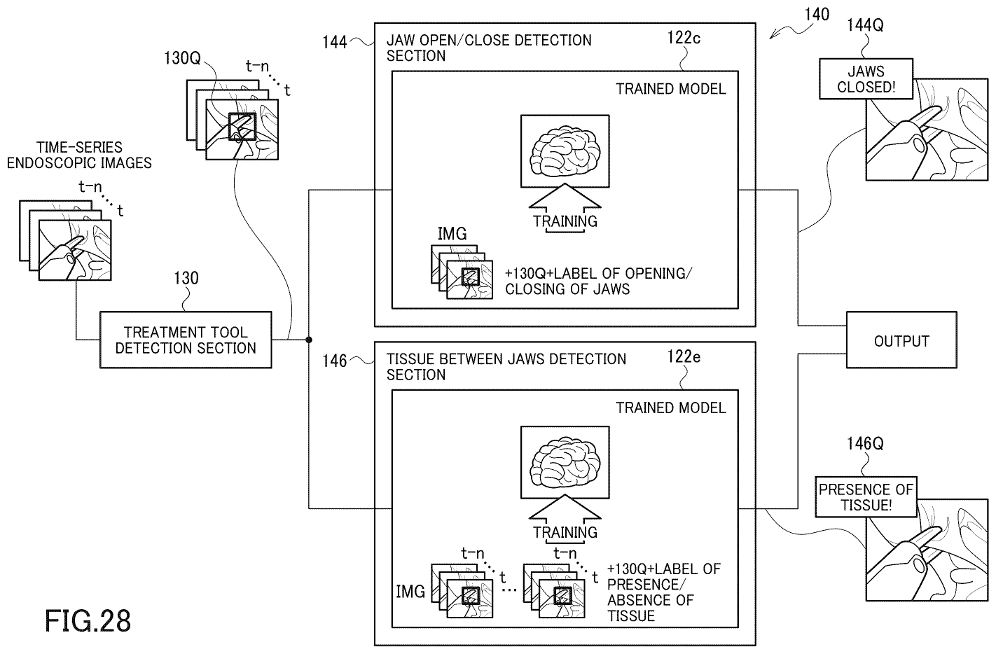

FIG. 28 is a third example configuration of the contact detection section in the second embodiment. The parts different from those in the first example configuration of FIG. 21 will mainly be described. The current frame is denoted as t and a nth frame before thereof is denoted as t−n.

First, the inference phase will be described. The treatment tool detection section 130 detects the treatment tool from an endoscopic image of a frame t, and outputs the detection result 130Q. The detection result 130Q is also output for the frames t−n, . . . , t−1 and stored in the memory 120, for example. The jaw open/close detection section 144 detects the open/closed state of the jaws from the endoscopic image of the frame t and outputs the detection result 144Q. The trained model 122 in FIG. 12 includes a trained model 122e for detection of a tissue between the jaws. The tissue between jaws detection section 146 inputs time-series endoscopic images of frames t−n, . . . , t−1, t and the detection result 130Q for each of the images to the trained model 122e. The trained model 122e detects the presence/absence of the tissue between the jaws from the input data, and outputs the detection result 146Q thereof. The trained model which handles time-series data includes, for example, a neural network using a Long Short Term Memory (LSTM) and the like. Note that the jaw open/close detection section 144 may also be configured to detect opening/closing of the jaws from the time-series endoscopic images.

As a result, in some embodiments, even if the situation is changed after a tissue grasping operation so as not to allow determination of the presence/absence of the tissue between the jaws, it is possible to recognize the grasp state from time-series information. By including time-series data to recognize the grasp state, it is possible to recognize the grasp or non-grasp state based on the previous detection result of grasping even if the presence/absence of the tissue between the jaws cannot be determined from the endoscopic image.

### 4. Third Embodiment

In the third embodiment, the treatment tool is an energy device. The energy device may be any of devices using high frequency power, ultrasound, or both. In addition, the energy device may be any of a bipolar device or a monopolar device. In the third embodiment, contact detection from an image and contact detection based on electrical information of the energy device are integrated to detect the contact state between the treatment tool and the tissue. Note that the description of the parts similar to those in the first or second embodiment are omitted. In the third embodiment, for example, the configuration of the medical system 1, the configuration of the controller 100, and the processing performed by the treatment tool detection section 130 are similar to those in the first embodiment.

First, a generator 235 which drives an energy device 236 will be described. The generator 235 supplies energy to the energy device 236, controls the energy supply, and acquires electrical information. In other words, the generator 235 outputs high frequency power, and the energy device 236 outputs the high frequency power from an end effector. Alternatively, the end effector of the energy device 236 has an ultrasonic device. The generator 235 outputs a drive signal of the ultrasonic device, and the ultrasonic device of the end effector receives the drive signal to output ultrasound.

The electrical information is electrical impedance obtained when the energy device 236 outputs high frequency power to a tissue. Note that the electrical information is not limited thereto. The electrical information may be current, voltage, or a phase between current and voltage. Alternatively, the electrical information may be power, amount of power, impedance, resistance, reactance, admittance (reciprocal of impedance), conductance (real part of admittance), or susceptance (imaginary part of admittance). Alternatively, the electrical information may be the change over time as described above, change between parameters, calculus between parameters (when P is a parameter, a differential over time is dP/dt, and a differential due to resistance is dP/dR), values derived by elementary arithmetic such as sum difference for each cluster, or trigger information such as whether or not each threshold is crossed.

Alternatively, the electrical information is mechanical impedance obtained when the energy device 236 outputs ultrasound to a tissue. However, the electrical information is not limited thereto. The electrical information may be change over time in the mechanical impedance, or the trigger information such as whether or not each threshold is crossed.

Hereinafter, the electrical information is assumed to be electrical impedance or mechanical impedance and is simply referred to as impedance. The impedance in the following description can be read as electrical information.

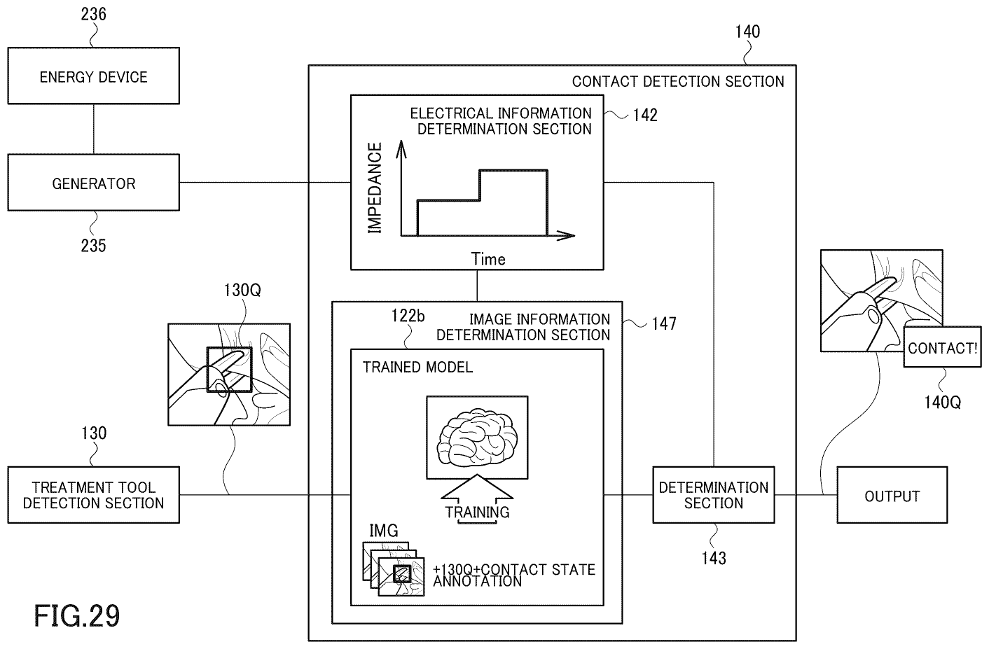

FIG. 29 is a first example configuration of the contact detection section in the third embodiment. The contact detection section 140 includes an image information determination section 147, an electrical information determination section 142, and a determination section 143.

The image information determination section 147 uses the trained model 122b for contact detection to detect the contact state between the treatment tool and the tissue from the endoscopic image. This technique of contact detection from an image is as described in the first embodiment. Alternatively, as the technique of contact detection from an image, the technique described in the second embodiment can be employed.

When the image information determination section 147 determines that the treatment tool is in contact with the tissue, the electrical information determination section 142 acquires impedance from the generator 235 and determines the contact state between the treatment tool and the tissue based on the impedance. When the jaws are grasping the tissue or a monopolar device is in contact with the tissue, the impedance according to the tissue will be observed. This allows determination of contact or non-contact based on the impedance. As one example, the electrical information determination section 142 determines whether or not the impedance is equal to or greater than a specified value, thereby making determination of contact or non-contact. The electrical information determination section 142 may be realized by a rule-based program or a trained model using machine learning.

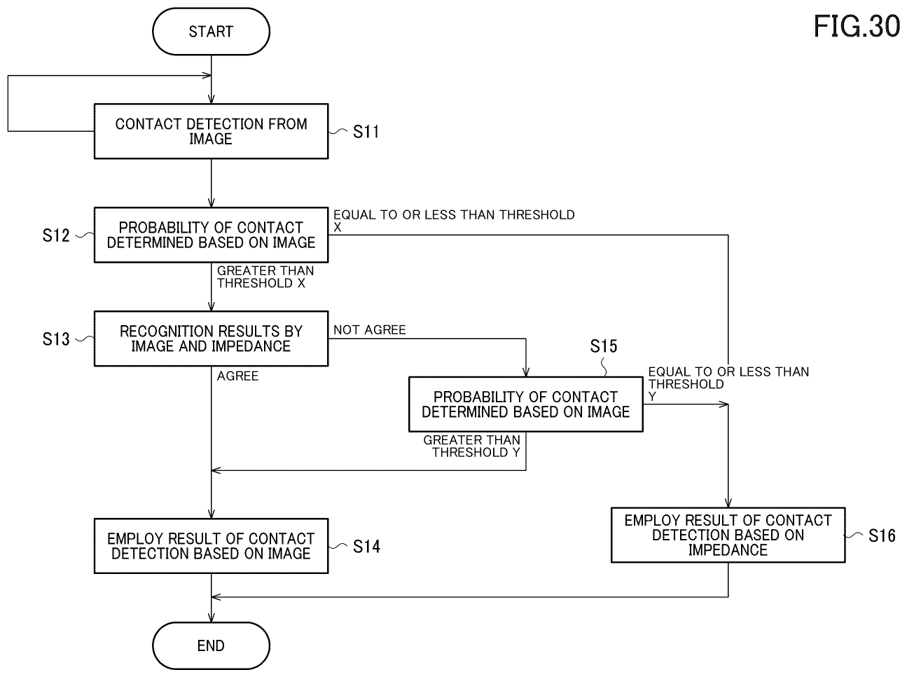

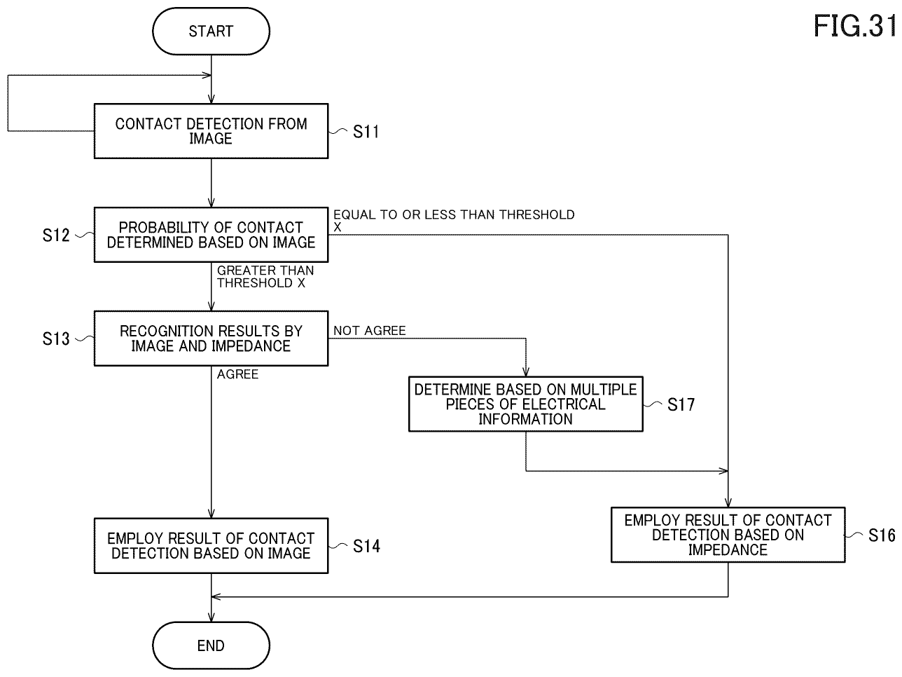

The determination section 143 integrates the detection result by the image information determination section 147 and the detection result by the electrical information determination section 142 to detect the contact state between the treatment tool and the tissue, and outputs the detection result 140Q. The integration processing will be described in the flows in FIGS. 30 and 31.

As a result, in some embodiments, since the contact detection is performed by a combination of the image and the impedance, it is expected to improve the accuracy of the contact detection as compared to the contact detection only from the image. In addition, when the contact detection is performed using only electrical or load information, the contact detection can only be initiated at timing when a handpiece is switched on. Therefore, action after detecting contact will be delayed. The action is, for example, tissue type recognition or automatic energy adjustment based thereon, etc. Delay in such action reduces the advantage of the contact detection. As a result, in some embodiments, the contact detection from the image is also performed so that the action after the contact detection can be accelerated.

FIG. 30 is a first example flow of processing performed by the contact detection section in the third embodiment. In this flow, when the trained model 122b of the image information determination section 147 determines that the treatment tool is in contact with the tissue, the determination section 143 classifies cases according to a probability of being determined as contact.

In the step S11, the image information determination section 147 uses the endoscopic image to perform contact detection. This processing is performed to the endoscopic image of each frame.

When the treatment tool is determined to be in contact with the tissue in the step S11, the determination section 143 compares a probability of contact output by the trained model 122b of the image information determination section 147 with a threshold x in the step S12. An example of the threshold x is 60%, but may be arbitrary.

When the probability is equal to or less than the threshold x in the step S12, the electrical information determination section 142 performs contact detection using the impedance in the step S16. The determination section 143 employs the detection result of the contact state output by the electrical information determination section 142 as the final detection result 140Q.

When the probability is greater than the threshold x in the step S12, the electrical information determination section 142 performs contact detection using the impedance in the step S13. The determination section 143 compares the detection result output by the image information determination section 147 with the detection result output by the electrical information determination section 142.

When the two detection results agree with each other in the step S13, the determination section 143 employs the detection result of the contact state output by the image information determination section 147 as the final detection result 140Q in the step S14.

When the two detection results do not agree with each other in the step S13, the determination section 143 compares the probability of contact output by the trained model 122b of the image information determination section 147 with a threshold y in the step S15. An example of the threshold y is 95%, but it may be an arbitrary value greater than the threshold x.

When the probability is equal to or less than the threshold y in the step S15, the determination section 143 employs the detection result of the contact state output by the electrical information determination section 142 as the final detection result 140Q in the step S16.

When the probability is greater than the threshold y in the step S15, the determination section 143 employs the detection result of the contact state output by the image information determination section 147 as the final detection result 140Q in the step S14.

FIG. 31 is a second example flow of processing performed by the contact detection section in the third embodiment. In this flow, when the results of contact detection from the image and the impedance are different from each other, the electrical information to be used for detection will be increased. Utilizing multiple pieces of electrical information, rather than only a single piece of impedance information, improves the detection accuracy In addition, utilizing multiple pieces of electrical information only when necessary makes the recognition flow efficient.

The parts different from those in the first flow in FIG. 30 will be described. When the two detection results do not agree with each other in the step S13, the electrical information determination section 142 determines the contact state between the treatment tool and the tissue based on the multiple pieces of electrical information in the step S17. As one example, the multiple pieces of electrical information corresponds to impedance, impedance variation, and a current value. In this case, when the impedance is less than a specified value, the impedance variation is less than a specified value, and the current value is equal to or greater than a specified value, the electrical information determination section 142 determines that the treatment tool is in contact with the tissue. In the step S16 after the step S17, the determination section 143 employs the detection result of the contact state output by the electrical information determination section 142 as the final detection result 140Q.

In the present flow, in a normal case, the electrical information determination section 142 performs contact detection by determining whether or not the impedance is equal to or greater than the specified value. The normal case refers to processing from S12 to S16 or processing from S12 through S13 to S14. Further, only in processing from S12 through S13 and S17 to S16 in which the two detection results are determined as not agreeing with each other, the electrical information determination section 142 uses the multiple pieces of electrical information to perform contact detection.

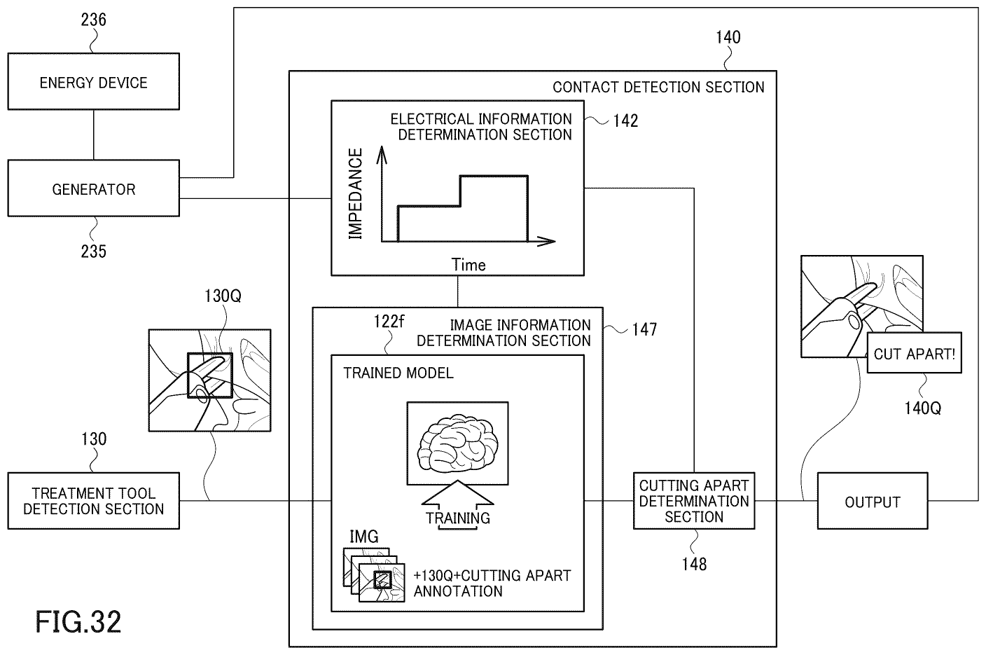

FIG. 32 is a second example configuration of the contact detection section in the third embodiment. The contact detection section 140 includes the image information determination section 147, the electrical information determination section 142, and a cutting apart determination section 148.

The trained model 122 in FIG. 2 includes a trained model 122f for detecting cutting apart of a tissue from an image. First, the inference phase will be described.

The image information determination section 147 inputs the endoscopic image and the detection result 130Q by the treatment tool detection section 130 to the trained model 122f, or inputs the image of the region of interest of the endoscopic image, which is set based on the detection result 130Q, to the trained model 122f. The trained model 122f detects cutting apart of the tissue from the input data.

The electrical information determination section 142 uses a function of the generator 235 for detecting cutting apart to obtain a detection result of cutting apart. Alternatively, the electrical information determination section 142 may acquire the impedance from the generator 235 and detect cutting apart of the tissue from the impedance. This cutting apart detection may be performed only when the image information determination section 147 determines that the treatment tool is in contact with the tissue, or may be performed all the time. As an example, the energy device is an ultrasonic device. The generator 235 may detect a load fluctuation of an ultrasonic probe that occurs at the time of cutting apart of the tissue, and the electrical information determination section 142 may acquire the detection result of cutting apart of the tissue.

The cutting apart determination section 148 integrates the detection result of cutting apart output by the image information determination section 147 and the detection result of cutting apart output by the electrical information determination section 142 to output the final detection result 140Q of cutting apart. The processor 110 performs output using the detection result 140Q. For example, when the tissue is determined to be cut apart, the processor 110 transmits an instruction for suppressing or stopping energy output to the generator 235, and the generator 235 suppresses or stops the energy output.

Next, the training phase of the trained model 122f will be described. The training system trains a model to detect cutting apart of the tissue from input data, thereby generating the trained model 122f. The input data corresponds to the endoscopic image IMG and the annotation indicating the position or the region of the treatment tool. Alternatively, the input data is the image of the region of interest of the endoscopic image IMG, which is set based on the annotation. The training data corresponds to a plurality of endoscopic images IMG, the annotation indicating the position or the region of the treatment tool in each of the images, and a label indicating a cut apart state of the tissue in each image. The label indicating the cut apart state of the tissue is, for example, a label indicating a state before cutting apart and a label indicating a state after cutting apart.

The cutting apart detection function based on the impedance described above is affected by a condition of a biological tissue, a type of a biological tissue, the way of grasping a handle, and the way of grasping a tissue. In addition, there may be a scene in which an image alone is not sufficient to allow detection of cutting apart due to the effect of tissue adhesion or the like. By using a combination of cutting apart detection based on the electrical information and cutting apart detection from an image, the frequency of detection omission can be reduced. The effect is to reduce excessive temperature rise of a device after tissue incision and to reduce the risk of damage to a probe or a tissue pad.

In the above techniques and the first to third embodiments, the medical system 1 includes the memory 120 which stores the trained model 122, and the processor 110. The trained model 122 is a model trained by training data including the endoscopic image and the contact state between the treatment tool and the tissue in the endoscopic image. The endoscopic image is captured by the endoscope, which captures an image including the treatment tool and the tissue to be treated. The processor 110 acquires the endoscopic image including the treatment tool and the tissue. The processor 110 uses the trained model to detect, from the endoscopic image, the contact state between the treatment tool and the tissue as first information.

As a result, in some embodiments, it is possible to detect the contact state between the treatment tool and the tissue from the endoscopic image, and use the detection result to provide various support to a practitioner. For example, it is possible to present the support information such as information about a contacted tissue to the practitioner only when the treatment tool comes in contact with the tissue. This can prevent information presentation at unnecessary timing for the practitioner and reduce annoyance. In addition, it is possible to detect the contact state between the treatment tool and the tissue by recognition processing using an image, and thus it is possible to recognize the contact state without increasing the size of the treatment tool while reducing the device cost as compared to a case where a tactile sensor and the like is provided on the treatment tool. Further, the contact detection from an image does not limit a device used as the treatment tool, so that it is applicable to a variety of existing treatment tools.

Further in the present embodiment, the processor 110 may detect the region of interest including the treatment tool from the endoscopic image. The processor 110 may use the trained model 122 to detect, from the image of the region of interest of the endoscopic image, the contact state between the treatment tool and the tissue. Note that the setting of the region of interest is described in FIGS. 13-18, etc.

Among the tissues, it is the surrounding tissue around the treatment tool in contact with the tissue that is subjected to the action of the treatment tool. For example, there is high correlation between the movement vectors of the treatment tool and the surrounding tissue. Alternatively, it is the surrounding tissue around the treatment tool that is affected by energy treatment. As a result, in some embodiments, the region of interest including the treatment tool is detected and the contact state is detected from the image of the region of interest, thereby allowing accurate detection of the contact state using the tissue around the treatment tool, which is subjected to the action of the treatment tool.

Further in the present embodiment, the processor 110 may perform support processing associated with treatment using the treatment tool when detecting the first information indicating that the treatment tool is in contact with the tissue.

Further in the present embodiment, the support processing may be processing of presenting the first information, processing of presenting the support information regarding the treatment, processing of presenting the support information regarding the energy output setting of the treatment tool which is an energy device, or processing of automatic control of the treatment tool which is an energy device. Note that an example of the support information is described in FIG. 4, etc.

As a result, in some embodiments, it is possible to detect, from the endoscopic image, the contact state between the treatment tool and the tissue, and use the detection result to provide various support to the practitioner. The “support information regarding the treatment” is, for example, a type or a condition of a tissue in contact with the treatment tool. The “support information regarding the energy output setting” is, for example, a recommended output setting determined based on an image and the like. The “processing of automatic control of the treatment tool” is, for example, processing of changing, suppressing, or stopping energy output. Note that the support processing may be support using the detection result of the contact state, and not limited to the examples mentioned herein.

Further in the present embodiment, the processor 110 may obtain, from the endoscopic image, the average movement vector of the treatment tool and the average movement vector of the surrounding tissue which is the tissue around the treatment tool. The processor 110 may use the trained model 122 to detect, from the average movement vectors of the treatment tool and the surrounding tissue, the contact state between the treatment tool and the tissue. The contact detection using movement vectors is described in FIGS. 5-11, etc.

As described in FIG. 6, etc., when the treatment tool is in contact with the tissue, the correlation between the movement vectors of the treatment tool and the tissue is high, and when the treatment tool is not in contact with the tissue, the correlation between the movement vectors of the treatment tool and the tissue is low. As a result, in some embodiments, it is possible to detect the contact state between the treatment tool and the tissue by utilizing this correlation.

Further in the present embodiment, the processor 110 may use at least one of the following (i), (ii), (iii), and (iv) to detect the contact state between the treatment tool and the tissue. (i) The average movement vector of the treatment tool. (ii) The average movement vector of the surrounding tissue. (iii) The Euclidean distance between the average movement vectors of the treatment tool and the surrounding tissue. (iv) The direction difference between the average movement vectors of the treatment tool and the surrounding tissue. These techniques are described in FIGS. 7-9, etc.

Further in the present embodiment, the processor 110 may decompose the movement vector of the surrounding tissue into the parallel component and the vertical component relative to the movement direction of the treatment tool, and use statistics of the parallel component and the vertical component to detect the contact state between the treatment tool and the tissue. Such technique is described in FIGS. 10-11, etc.

As a result, in some embodiments, it is possible to use the average movement vector or a quantity calculated from the average movement vector to evaluate the correlation between the movement vectors of the treatment tool and the tissue, thereby detecting the contact state between the treatment tool and the tissue.

Further in the present embodiment, the treatment tool may be an energy device that treats a tissue by outputting energy from an end effector. The processor 110 may use the first information indicating the contact state determined based on the endoscopic image and second information indicating the contact state determined based on the electrical information on the energy output to determine the contact state between the treatment tool and the tissue. Such technique is described in FIGS. 29-32, etc.

As a result, in some embodiments, since the contact detection is performed by combining the contact detection from the image and the contact detection based on the electrical information, it is expected to improve the accuracy of the contact detection as compared to the contact detection based on only one of them.

Further in the present embodiment, determination of the contact state may be determination of whether or not the treatment tool is in contact with the tissue. The processor 110 may decide whether to employ the first information or the second information depending on an estimation probability of determination that the treatment tool is in contact with the tissue in estimation of the first information using the trained model 122. Such technique is described in FIGS. 30-31, etc.

As a result, in some embodiments, it is possible to decide whether to employ the result of contact detection from the image or the result of contact detection based on the electrical information depending on the estimation probability of the contact detection from the image. For example, when the estimation probability of the contact detection from the image is high, it is possible to employ the result of the contact detection from the image. When the estimation probability of the contact detection from the image is high, it is possible to decide whether to employ the result of the contact detection from the image or the result of the contact detection based on the electrical information while taking into consideration the result of the contact detection based on the electrical information.

Further in the present embodiment, when the first information does not agree with the second information, the processor 110 may determine the contact state based on the multiple pieces of electrical information on the energy output. Such technique is described in FIG. 31, etc.

As a result, in some embodiments, when the result of the contact detection from the image does not agree with the result of the contact detection based on the electrical information, the estimation probability of such contact detection is assumed to be low. In such case, it is possible to accurately detect the contact state by determining the contact state based on the multiple pieces of electrical information.

Further in the present embodiment, the treatment tool may have a pair of openable/closable jaws. The memory 120 may store a first trained model and a second trained model as the trained model 122. The first trained model may be a model trained to detect opening/closing of the jaws from the endoscopic image. The second trained model may be a model trained to detect the presence/absence of the tissue between the jaws from the endoscopic image. The processor 110 may use the first trained model to perform first detection that detects opening/closing of the jaws from the endoscopic image. The processor 110 may use the second trained model to perform second detection that detects, from the endoscopic image, the presence/absence of the tissue between the jaws. The processor 110 may detect the contact state between the treatment tool and the tissue based on the results of the first detection and the second detection. Such technique is described in FIGS. 21-28, etc. In the example configurations of FIGS. 21 and 26, the trained model 122c is the first trained model and the trained model 122d is the second trained model. In the example configuration of FIG. 28, the trained model 122c is the first trained model and the trained model 122e is the second trained model.

As a result, in some embodiments, the trained model of contact detection is separated into the first trained model for detecting opening/closing of the jaws and the second trained model for detecting the tissue between the jaws. This achieves higher estimation accuracy of each model than that of estimation of the contact detection with one model, allowing contact detection with higher accuracy.

Further in the present embodiment, when it is detected that the jaws are closed in the first detection and there is a tissue between the jaws in the second detection, the processor 110 may determine that the treatment tool is in contact with the tissue.

Further in the present embodiment, a condition for determining that there is a tissue between the jaws in the second detection is that one of a pair of openable/closable jaws is covered with the tissue and the other is not covered with the tissue.

By using these determination conditions, it is possible to determine, based on the detection result of opening/closing of the jaws and the detection result of the tissue between the jaws, whether or not the jaws of the treatment tool are grasping the tissue, i.e. whether or not the treatment tool is in contact with the tissue.

Further in the present embodiment, the treatment tool may be an energy device that treats a tissue by outputting energy from an end effector. The processor 110 may present the output setting of energy output when detecting the first information indicating the contact between the treatment tool and the tissue. The processor 110 may cause the energy device to output energy with the above output setting when receiving an output instruction after the presentation. Such technique is described in FIGS. 1 and 4, etc.

As a result, in some embodiments, it is possible to present the support information such as information about a contacted tissue to the practitioner only when the treatment tool comes in contact with the tissue, and request input for acceptance or rejection thereof. This can prevent information presentation at unnecessary timing for the practitioner and reduce annoyance.

Further, in the present embodiment, the treatment tool may be an energy device that treats a tissue by outputting energy from an end effector. The processor 110 may make determination on setting change of the energy output when detecting the first information indicating that the treatment tool and the tissue are changed from contact to non-contact. The detection of cutting apart of the tissue is described in FIGS. 19 and 20, etc. The “determination on setting change of the energy output” is, for example, determination of whether or not to change, suppress, or stop the energy output when the tissue is cut apart.

As a result, in some embodiments, by detecting that the treatment tool and the tissue are changed from contact to non-contact, it is possible to detect that the tissue is cut apart by energy treatment. By making determination on setting change of the energy output when there is no contact between the treatment tool and the tissue, it is possible to support the practitioner.

Further, the present embodiment may be implemented as a method for detecting a contact state as described below. In other words, the method for detecting a contact state includes capturing an endoscopic image by an endoscope, the endoscopic image including a treatment tool and a tissue to be treated. The method for detecting a contact state includes detecting a contact state between the treatment tool and the tissue from the endoscopic image using a trained model. The trained model is a model trained by training data including the endoscopic image and the contact state between the treatment tool and the tissue in the endoscopic image.

### 5. Fourth Embodiment

In the fourth embodiment, the treatment tool is an energy device. The energy device may be any of devices using high frequency power, ultrasound, or both. In addition, the energy device may be any of a bipolar device or a monopolar device. In the fourth embodiment, by combining detection of opening/closing of jaws from an image and detection of a tissue between jaws based on the electrical information, the contact state between the treatment tool and the tissue is to be detected. Note that the description of the parts similar to those in the first, second, and third embodiments are omitted. In the fourth embodiment, for example, the configuration of the medical system 1, the configuration of the controller 100, and the processing performed by the treatment tool detection section 130 are similar to those in the first embodiment.

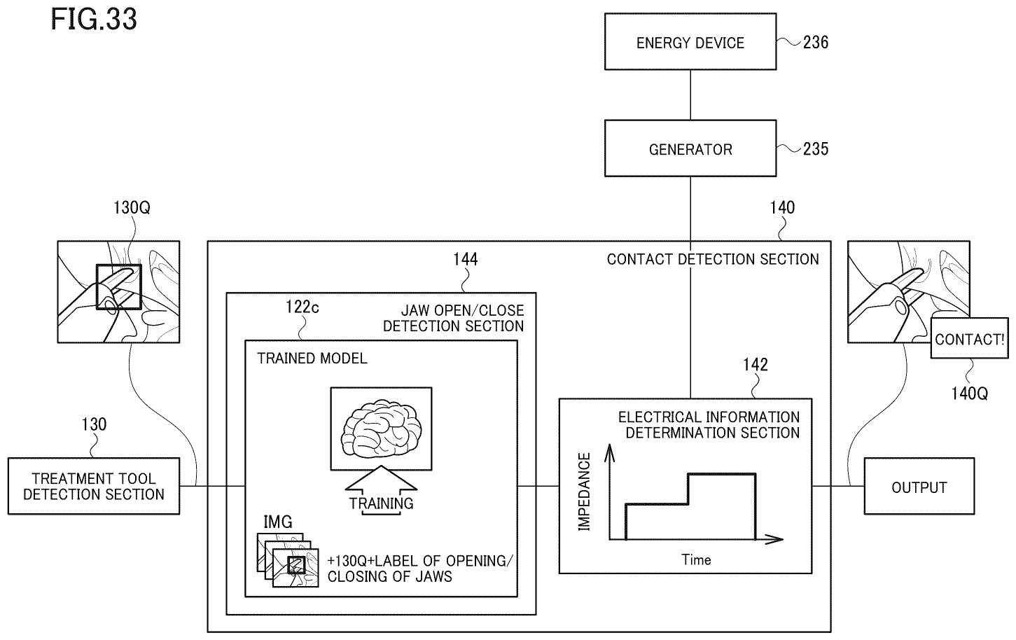

FIG. 33 is an example configuration of the contact detection section in the fourth embodiment. The contact detection section 140 includes the jaw open/close detection section 144 and the electrical information determination section 142.

The jaw open/close detection section 144 uses the trained model 122c to detect, from the endoscopic image, the open/closed state of the jaws captured in the endoscopic image. This detection technique is as described in FIG. 21.

When the jaw open/close detection section 144 determines that the jaws are closed, the electrical information determination section 142 acquires the electrical information from the generator 235, uses the electrical information to detect the contact state between the treatment tool and the tissue, and outputs the detection result 140Q. Specifically, the electrical information determination section 142 uses the electrical information to detect the presence/absence of a tissue between the jaws. This detection technique is as described in the third embodiment.

When the electrical information determination section 142 determines that there is a tissue between the jaws, the contact detection section 140 determines that the treatment tool is grasping the tissue, i.e. the treatment tool is in contact with the tissue.

In the fourth embodiment below, the medical system 1 includes the memory 120 which stores the trained model 122, and the processor 110. The trained model 122 is a model trained by training data. The training data includes the endoscopic image captured by the endoscope which captures an image including the treatment tool having openable/closable jaws and a treatment target tissue, and an open/closed state of the jaws in the endoscopic image. The treatment tool is an energy device that treats a tissue by outputting energy from the jaws. The processor 110 acquires the endoscopic image including the treatment tool and the tissue. The processor 110 performs the first detection that detects opening/closing of the jaws from the endoscopic image using the trained model. The processor 110 performs the second detection that detects the presence/absence of a tissue between the jaws based on the electrical information on the energy output. The processor 110 detects, based on the results of the first detection and the second detection, a grasped state of the tissue by the jaws as the contact state.

When considering recognition from the image, it is assumed that opening/closing of the jaws is easier to detect than the contact between the treatment tool and the tissue. As a result, in some embodiments, by combining the detection of opening/closing of the jaws, which is considered to be more accurate than the detection of the contact state, with the detection of the tissue between the jaws based on the impedance, it is possible to accurately detect the contact state.

Further in the present embodiment, when detecting that the jaws are closed in the first detection, the processor 110 performs the second detection.

As a result, in some embodiments, only the detection of opening/closing of the jaws from the image will be performed until the jaws are detected as closed, and when the jaws are detected as closed, detection of the tissue between the jaws based on the impedance will be performed. This can reduce a computational burden.

Further in the present embodiment, when detecting that the jaws are closed in the first detection and there is a tissue between the jaws in the second detection, the processor 110 determines that the jaws are grasping the tissue.

By using such determination conditions, it is possible to determine, based on the detection result of opening/closing of the jaws and the detection result of the tissue between the jaws, whether or not the jaws of the treatment tool are grasping the tissue, i.e. whether or not the treatment tool is in contact with the tissue.

Further, the present embodiment may be implemented as a method for detecting a contact state. In other words, the method for detecting a contact state is a method of detecting a contact state between a treatment tool and a tissue to be treated. The treatment tool has openable/closable jaws, and is an energy device that treats a tissue by outputting energy from the jaws. The method for detecting a contact state includes capturing an endoscopic image by an endoscope, the endoscopic image including the treatment tool and the tissue to be treated. The method for detecting a contact state includes performing the first detection that detects opening/closing of the jaws from the endoscopic image using the trained model 122. The trained model 122 is a model trained by training data including the endoscopic image and the open/closed state of the jaws in the endoscopic image. The method for detecting a contact state includes performing the second detection that detects the presence/absence of a tissue between the jaws based on the electrical information on the energy output. The method for detecting a contact state includes detecting the grasped state of the tissue by the jaws as the contact state based on the results of the first detection and the second detection.

Although the embodiments to which the present disclosure is applied and the modifications thereof have been described above, the present disclosure is not limited to the embodiments and the modifications thereof, and various modifications and variations in components may be made in implementation without departing from the spirit and scope of the present disclosure. In addition, the plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Further, any term cited with a different term having a broader meaning or the same meaning at least once in the specification and the drawings can be replaced by the different term in any place in the specification and the drawings.

1. A medical system comprising:
a memory that stores a trained model trained by training data, the training data including an endoscopic image captured by an endoscope that captures an image including a treatment tool and a tissue to be treated, and a contact state between the treatment tool and the tissue in the endoscopic image; and
a processor,
wherein the processor is configured to:
acquire the endoscopic image including the treatment tool and the tissue; and
detect, from the endoscopic image, the contact state between the treatment tool and the tissue as first information by using the trained model.

2. The medical system as defined in claim 1, wherein
the processor detects, from the endoscopic image, a region of interest including the treatment tool, and detects the contact state between the treatment tool and the tissue from an image of the region of interest of the endoscopic image by using the trained model.

3. The medical system as defined in claim 1, wherein
the processor performs support processing associated with treatment using the treatment tool when detecting the first information indicating that the treatment tool is in contact with the tissue.

4. The medical system as defined in claim 3, wherein
the support processing is processing of presenting the first information, processing of presenting support information regarding the treatment, processing of presenting support information regarding an energy output setting of the treatment tool which is an energy device, or processing of automatic control of the treatment tool which is an energy device.

5. The medical system as defined in claim 1, wherein the processor:
obtains, from the endoscopic image, an average movement vector of the treatment tool and an average movement vector of a surrounding tissue which is the tissue around the treatment tool; and
detects the contact state between the treatment tool and the tissue from the average movement vector of the treatment tool and the average movement vector of the surrounding tissue by using the trained model.

6. The medical system as defined in claim 5, wherein
the processor detects the contact state between the treatment tool and the tissue using at least one of the average movement vector of the treatment tool, the average movement vector of the surrounding tissue, Euclidean distance between the average movement vector of the treatment tool and the average movement vector of the surrounding tissue, and direction difference between the average movement vector of the treatment tool and the average movement vector of the surrounding tissue.

7. The medical system as defined in claim 5, wherein
the processor decomposes the movement vector of the surrounding tissue into a parallel component and a vertical component relative to a movement direction of the treatment tool, and detects the contact state between the treatment tool and the tissue using statistics of the parallel component and the vertical component.

8. The medical system as defined in claim 1, wherein
the treatment tool is an energy device that treats the tissue by outputting energy from an end effector, and
the processor determines the contact state between the treatment tool and the tissue using the first information indicative of the contact state determined based on the endoscopic image and second information indicative of the contact state determined based on electrical information on the energy output.

9. The medical system as defined in claim 8, wherein
the determination of the contact state is determination of whether or not the treatment tool is in contact with the tissue, and
the processor decides whether to employ the first information or the second information depending on an estimation probability of determination that the treatment tool is in contact with the tissue in estimation of the first information using the trained model.

10. The medical system as defined in claim 8, wherein
the processor determines the contact state based on multiple pieces of electrical information on the energy output when the first information does not agree with the second information.

11. The medical system as defined in claim 1, wherein
the treatment tool has a pair of openable/closable jaws,
the memory stores, as the trained model, a first trained model trained to detect opening/closing of the jaws from the endoscopic image, and a second trained model trained to detect presence/absence of the tissue between the jaws from the endoscopic image, and
the processor:
performs first detection that detects opening/closing of the jaws from the endoscopic image using the first trained model;
performs second detection that detects presence/absence of a tissue between the jaws from the endoscopic image using the second trained model; and
detects, based on results of the first detection and the second detection, the contact state between the treatment tool and the tissue.

12. The medical system as defined in claim 11, wherein
the processor determines that the treatment tool is in contact with the tissue when it is detected that the jaws are closed in the first detection and there is the tissue between the jaws in the second detection.

13. The medical system as defined in claim 12, wherein
a condition for determining that there is the tissue between the jaws in the second detection is that one of a pair of openable/closable jaws is covered with the tissue and the other is not covered with the tissue.

14. The medical system as defined in claim 1, wherein
the treatment tool is an energy device that treats the tissue by outputting energy from an end effector, and
the processor presents an output setting of the energy output when detecting the first information indicative of contact between the treatment tool and the tissue, and causes the energy device to perform the energy output with the output setting when receiving an output instruction after the presentation.

15. The medical system as defined in claim 1, wherein
the treatment tool is an energy device that treats the tissue by outputting energy from an end effector, and
the processor makes determination on setting change of the energy output when detecting the first information indicating that the treatment tool and the tissue are changed from contact to non-contact.

16. A method for detecting a contact state, comprising:
capturing an endoscopic image including a treatment tool and a tissue to be treated by an endoscope;
detecting, from the endoscopic image, the contact state between the treatment tool and the tissue as first information using a trained model trained by training data, the training data including the endoscopic image and the contact state between the treatment tool and the tissue in the endoscopic image.

17. A medical system comprising:
a memory that stores a trained model trained by training data, the training data including an endoscopic image captured by an endoscope that captures an image including a treatment tool with an openable/closable jaw and a tissue to be treated, and an open/closed state of the jaw in the endoscopic image; and
a processor,
wherein the treatment tool is an energy device that treats the tissue by outputting energy from the jaw, and
the processor is configured to:
acquire the endoscopic image including the treatment tool and the tissue;
perform first detection that detects opening/closing of the jaw from the endoscopic image using the trained model;
perform second detection that detects presence/absence of a tissue between the jaw based on electrical information on the energy output; and
detect, based on results of the first detection and the second detection, a grasped state of the tissue by the jaw as a contact state.

18. The medical system as defined in claim 17, wherein
the processor performs the second detection when detecting that the jaw is closed in the first detection.

19. The medical system as defined in claim 17, wherein
the processor determines that the jaw is grasping the tissue when detecting that the jaw is closed in the first detection and detecting that there is the tissue between the jaw in the second detection.

20. A method for detecting a contact state that detects a contact state between a treatment tool and a tissue to be treated, the treatment tool having an openable/closable jaw and treating the tissue by outputting energy from the jaw, the method comprising:
capturing an endoscopic image including the treatment tool and the tissue by an endoscope;
performing first detection that detects opening/closing of the jaw from the endoscopic image using a trained model trained by training data, the training data including the endoscopic image and an open/closed state of the jaw in the endoscopic image;
performing second detection that detects, based on electrical information on the energy output, presence/absence of a tissue between the jaw; and
detecting, based on results of the first detection and the second detection, a grasped state of the tissue by the jaw as the contact state.
